(19) United States
(12) Patent Application Publication
(10) Pub. No.: US 2026/0234695 A1
(43) Pub. Date: Aug. 13, 2026

(54) COMPOSITIONS AND METHODS FOR PROBING RNA TERTIARY STRUCTURES

(71) Applicant: YALE UNIVERSITY, New Haven, CT (US)

(72) Inventors: Anna Marie Pyle, New Haven, CT (US); Shivali Patel, New Haven, CT (US); Matthew Simon, New Haven, CT (US)

(21) Appl. No.: 19/156,127

(22) PCT Filed: Feb. 22, 2024
(86) PCT No.: PCT/US2024/016817

§ 371 Date: Aug. 13, 2025

Related U.S. Application Data
(60) Provisional application No. 63/486,793, filed on Feb. 24, 2023.

(51) Int. Cl. C12Q 1/6806

(52) U.S. Cl. CPC C12Q 1/6806

(57) ABSTRACT

Compact RNA structural motifs control many aspects of gene expression, but the art lacks methods for finding these structures in the vast expanse of multi-kilobase RNAs. To adopt specific 3-D shapes, many RNA modules must compress their RNA backbones together, bringing negatively charged phosphates into close proximity. This is often accomplished by recruiting multivalent cations (usually Mg²⁺), which stabilize these sites and neutralize regions of local negative charge. Coordinated lanthanide ions, such as terbium (III) (Tb³⁺), can substitute for Mg²⁺ at these sites, inducing efficient RNA cleavage, thereby revealing compact RNA 3-D modules. This study presents Tb-seq, a high-throughput sequencing method for detecting compact tertiary structures in large RNAs. Tb-seq detects sharp backbone turns found in RNA tertiary structures and RNP interfaces, providing a way to scan transcriptomes for stable structural modules and potential riboregulatory motifs.

### CROSS-REFERENCE TO RELATED APPLICATIONS

The present application claims priority under 35 U.S.C. § 119(e) to U.S. Provisional Patent Application No. 63/486,793, filed Feb. 24, 2023, which is incorporated herein by reference in its entirety.

### STATEMENT REGARDING FEDERALLY SPONSORED RESEARCH

This invention was made with government support under HG011868 and GM007223-45 awarded by National Institutes of Health. The government has certain rights in this invention.

### SEQUENCE LISTING

The ASCII text file named “047162-7428WO1 Seq Listing” created on Feb. 16, 2024, comprising 21.1 Kbytes, is hereby incorporated by reference in its entirety.

### BACKGROUND

RNAs can adopt complex folded motifs and higher-order 3-D structures, all of which can be essential across a variety of specific cellular processes. Many types of multi-kilobase RNA transcripts contain regions of tertiary structure that, either alone or in concert with protein partners, carry out biological function. However, identifying these regions of complex RNA structure remains challenging. Current structure prediction methods on long RNAs are unable to pinpoint regions containing stable RNA tertiary structure modules or complex protein binding sites from sequence alone. While biophysical techniques such as NMR, x-ray crystallography and cryo-EM are invaluable tools for the observation of RNA structure, they are time consuming and difficult to perform on a multi kilobase-length RNA that contains a mixture of both structured and flexible regions. As the understanding of RNA's biological functions becomes increasingly important, and interest in small molecule targeting of RNAs grows, it is vital to develop new tools for identifying regions of tertiary structure in long RNA molecules.

In recent years, chemical probing has become a powerful tool for studying RNA structure. Many important advances have improved the ability to identify single-stranded versus double-stranded nucleotides in RNA, and these data have primarily been used to infer secondary but not tertiary structures of RNA. Fewer methods have been developed to detect higher order structure and these protocols are limited to an assessment of solvent accessible regions or identification of long-range RNA-RNA base-pairs by cross-linking methods or statistical correlations in mutational profiling. The field would benefit from a readily adaptable, high throughput approach for identifying regions of local tertiary structure, which are often hallmarks of functional RNA motifs and riboregulatory elements.

Therefore, there is a need to develop compositions and methods for identifying regions of local tertiary structure within large RNAs and ribonucleoprotein (RNP) interfaces. The present invention addresses this need.

### SUMMARY

In some aspects, the present invention is directed to the following non-limiting embodiments:

### Method of Probing Tertiary Structure of RNA Molecule

In some embodiments, the present invention is directed to a method of probing tertiary structure of a ribonucleotide (RNA) molecule.

In some embodiments, the method comprises contacting the RNA molecule with terbium (III) (Tb³⁺) under conditions that allow for Tb³⁺-mediated cleavage of the RNA molecule, thus generating at least one RNA fragment.

In some embodiments, the method further comprises determining a sequence of the at least one RNA fragment.

In some embodiments, the method further comprises identifying a site of the Tb³⁺-mediated cleavage based on the determined sequence of the at least one RNA fragment.

In some embodiments, the method further comprises identifying a tertiary structure in the RNA molecule based on the identified Tb³⁺-mediated cleavage site.

In some embodiments, the site of the Tb³⁺-mediated cleavage is within or adjacent to the tertiary structure of the RNA molecule.

In some embodiments, the RNA molecule comprises at least 2-100.000 nucleotides.

In some embodiments, the sequence of the at least one RNA fragment is determined by capillary electrophoresis or a next generation sequencing (NGS) method.

In some embodiments, the at least one RNA fragment is sequenced directly, or the at least one RNA fragment is converted to a DNA molecule and then sequenced.

In some embodiments, the at least one RNA fragment is converted to a DNA molecule by a reverse transcriptase (RT) and then sequenced.

In some embodiments, the at least one RNA fragment is processed into a cDNA library before being sequenced.

In some embodiments, the at least one RNA fragment or the cDNA molecules in the cDNA library is/are attached with a barcoding sequence.

In some embodiments, the determining step comprises aligning the sequence of the cDNA molecules with the sequence of the RNA molecule.

In some embodiments, identifying the site of the Tb³⁺-mediated cleavage comprises calculating a stop probability of one or more nucleotide in the RNA molecule.

In some embodiments, a nucleotide having a stop probability equal to or higher than a predetermined value indicates that the nucleotide is at the site of the Tb³⁺-mediated cleavage.

In some embodiments, the stop probability per RNA molecule nucleotide is calculated as an abundance of stops for the RNA molecule nucleotide in relative to a sum of the total number of read-through events plus the number of stops for the RNA molecule nucleotide.

In some embodiments, the stop probability per RNA molecule nucleotide in the absence of Tb³⁺ contacting is subtracted from the stop probability per RNA molecule nucleotide, thus providing an RNA molecule nucleotide reactivity value.

In some embodiments, only the RNA molecule nucleotides having read-throughs equal to or greater than a predetermined number are further considered as potential Tb³⁺-mediated cleavage sites.

In some embodiments, the RNA molecule nucleotide reactivity value is normalized to a predetermined value of top percentile of the stop rates for the RNA molecule.

In some embodiments, the normalized RNA molecule nucleotide reactivity value is scaled at a predetermined value.

In some embodiments, the RNA molecule is contacted with two or more distinct Tb³⁺ concentrations, and the identified tertiary structure in the RNA molecule is based on one or more identified Tb³⁺-mediated cleavage sites that show dependence on Tb³⁺ concentrations.

In some embodiments, the method is performed separately with (a) the RNA molecule free of a binding partner, and (b) the RNA molecule in the presence of the binding partner.

In some embodiments, the binding partner is a protein.

In some embodiments, the RNA molecule free of the protein binding partner is generated by removing the protein with a proteolytic enzyme.

In some embodiments, the method identifies a tertiary structure induced by the binding partner and/or a tertiary structure disrupted by the binding partner in the RNA molecule, based on a change in a pattern of the Tb³⁺-mediated cleavage between (a) and (b).

### Kit

In some aspects, the present invention is directed to a kit for probing tertiary structure of an RNA molecule.

In some embodiments, the kit comprises Tb³⁺.

In some embodiments, the kit further comprises a buffer solution, or a concentrate or substantially pure form thereof, for dissolving the RNA molecule, or diluting a RNA molecule stock solution, and allowing the Tb³⁺ to cleave the RNA molecule in a tertiary structure dependent manner.

In some embodiments, the kit further comprises a manual instructing that the RNA molecule is to be cleaved in the buffer solution in the presence of the Tb³⁺.

In some embodiments, the Tb³⁺ is present in the buffer solution, and the concentration of the Tb³⁺ ranges from about 0.01 mM to about 200 mM in the buffer.

In some embodiments, the Tb³⁺ is provided as a Tb (III) salt or a concentrate thereof, and the manual instructs that the concentration of the Tb³⁺ is to be adjusted to the range from about 0.01 mM to about 200 mM in the buffer.

In some embodiments, the buffer solution, or the concentrate or the substantially pure form thereof, comprises a Good's buffer.

In some embodiments, a component in the buffer solution ranges from about 0.01 mM to about 200 mM.

In some embodiments, the manual instructs that the concentrate or substantially pure form of the buffer solution is to be prepared so that the component in a buffer solution has a concentration ranging from about 0.01 mM to about 200 mM.

In some embodiments, the kit further comprises a monovalent salt, a divalent salt, or combinations thereof.

In some embodiments, the concentration of the monovalent salt or the concentration of the divalent salt ranges from about 0.01 mM to about 1M.

In some embodiments, the kit further comprises an RNase inhibitor.

In some embodiments, the kit further comprises a component for preparing an RNA sample for sequencing, wherein the component comprises a barcoding nucleic acid, a ligase, a primer, a reverse transcriptase, a DNA polymerase, an RNase, or combinations thereof.

In some embodiments, the kit further comprises a detergent for lysing the cell.

In some embodiments, the kit further comprises a protease inhibitor, a proteolytic enzyme, or combinations thereof.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1E: Developing a sequencing-based approach to detect Tb³⁺ cleavage sites. FIG. 1A: 3D structure of group II intron with insert showing localization of metal ion in region of negative electrostatic potential region. FIG. 1B: Mechanism of Tb³⁺ mediated cleavage. FIG. 1C: Denaturing electrophoresis of ³²P-labeled aI5γ RNA probed at the indicated TbCl₃ concentrations. FIG. 1D: Primer extension electrophoresis of corresponding cDNA products from reverse transcription. FIG. 1E: Tb-seq library construction workflow. Tb³⁺ cleaved RNA is reverse transcribed with a gene specific RT primer containing a 5′ adapter handle. The resulting cDNA is 3′ adapter-ligated and PCR amplified with Illumina multiplex handles. Stop sites are processed using the RTEvents counter script.

FIGS. 2A-2B: Tb-seq of O.i. intron detects long range, evolutionarily conserved RNA-RNA interactions. The arrows (black and white) indicate the connectivity of the molecule). FIG. 2A: Secondary structure of the O.i. intron displaying sites of strong Tb³⁺ cleavage (red/dark gray). Long range RNA-RNA interactions are indicated by Greek letters. EBS1 and EBS2 correspond to exon binding sites. Gray nucleotides indicate a lack of sequencing data in this region. FIG. 2B: 3-D structure of O.i. intron showing sites of strong Tb³⁺ cleavage on the RNA backbone (red/dark gray highlight). Inserts showing close up view of two long range RNA-RNA interactions (ζ-ζ′ and λ-λ′) with nucleotides that display Tb³⁺ cleavage (red/dark gray) and hydrogen bonds (dashed lines).

FIGS. 3A-3B: Tb-seq of HCV IRES detects conserved L-shaped bend in stem loop II. FIG. 3A: Secondary structure of HCV 5′ UTR Stem loop (SL) II displaying sites of strong Tb³⁺ cleavage (red/dark gray). FIG. 3B: 3-D structure of SL2 showing sites of strong Tb³⁺ cleavage on the RNA backbone (red/dark gray highlight). The RNA sequence shown in FIG. 3A is also shown in the table below:

HCV 5′ UTR Stem loop (SL) II:
(SEQ ID NO: 12)
UCCCCUGUGAGGAACUACUGUCUUCACGCAGAAAGCGCCUAGCCAU
GGCGUUAGUAUGAGUGUCGUACAGCCUCCAGGCCC

FIGS. 4A-4B: Probing RNA-Protein interactions in human RNase P. FIG. 4A: 3-D structure of RNase P complexed with its protein components showing sites of strong Tb³⁺ cleavage on the RNA backbone (red highlight). Inserts show close up views of two regions containing RNA-Protein interactions with nucleotides that display Tb³⁺ cleavage labeled (red) and hydrogen bonds (dashed lines). FIG. 41B: A Tb reactivity profile for the first 150 nt of RNase P. Nucleotides indicated in green/gray become less reactive when probed in the absence of protein components whereas nucleotides indicated in blue/gray (marked with *) become more reactive. The RNA sequences shown in FIG. 4B is also shown in the table below:

RNase P P3:
GGGGCCACGAGCUGAGUGCGUCCUGUCACUCCACUCCCAUGUCCCU
(SEQ ID NO: 13)
RNase P P9:
CAGAGGCGGCCCUAACAGGGCUCUCCCUGAGCUUCGGGGAGGCG
(SEQ ID NO: 14)

FIG. 5: Tb-seq identifies novel structural modules in SARS-CoV-2. Cell lysate probing of the 5′ terminal of SARS-COV2. Secondary structure of the SARS-CoV-2 displaying sites of strong Tb³⁺ cleavage (red/dark gray). The arrows (black and white) indicate the connectivity of the molecule). Inserts showing close up views of two regions that display A Tb reactivities. The sequences shown in FIG. 5 are also listed in the table below

SARS-COV2 RNA residues 1-1400
AUUAAAGGUUUAUACCUUCCCAGGUAACAAACCAACCAACUUUCGAU
CUCUUGUAGAUCUGUUCUCUAAACGAACUUUAAAAUCUGUGUGGCUG
UCACUCGGCUGCAUGCUUAGUGCACUCACGCAGUAUAAUUAAUAACU
AAUUACUGUCGUUGACAGGACACGAGUAACUCGUCUAUCUUCUGCAG
GCUGCUUACGGUUUCGUCCGUGUUGCAGCCGAUCAUCAGCACAUCUA
GGUUUCGUCCGGGUGUGACCGAAAGGUAAGAUGGAGAGCCUUGUCCC
UGGUUUCAACGAGAAAACACACGUCCAACUCAGUUUGCCUGUUUUAC
AGGUUCGCGACGUGCUCGUACGUGGCUUUGGAGACUCCGUGGAGGAG
GUCUUAUCAGAGGCACGUCAACAUCUUAAAGAUGGCACUUGUGGCUU
AGUAGAAGUUGAAAAAGGCGUUUUGCCUCAACUUGAACAGCCCUAUG
UGUUCAUCAAACGUUCGGAUGCUCGAACUGCACCUCAUGGUCAUGUU
AUGGUUGAGCUGGUAGCAGAACUCGAAGGCAUUCAGUACGGUCGUAG
UGGUGAGACACUUGGUGUCCUUGUCCCUCAUGUGGGCGAAAUACCAG
UGGCUUACCGCAAGGUUCUUCUUCGUAAGAACGGUAAUAAAGGAGCU
GGUGGCCAUAGUUACGGCGCCGAUCUAAAGUCAUUUGACUUAGGCGA
CGAGCUUGGCACUGAUCCUUAUGAAGAUUUUCAAGAAAACUGGAACA
CUAAACAUAGCAGUGGUGUUACCCGUGAACUCAUGCGUGAGCUUAAC
GGAGGGGCAUACACUCGCUAUGUCGAUAACAACUUCUGUGGCCCUGA
UGGCUACCCUCUUGAGUGCAUUAAAGACCUUCUAGCACGUGCUGGUA
AAGCUUCAUGCACUUUGUCCGAACAACUGGACUUUAUUGACACUAAG
AGGGGUGUAUACUGCUGCCGUGAACAUGAGCAUGAAAUUGCUUGGUA
CACGGAACGUUCUGAAAAGAGCUAUGAAUUGCAGACACCUUUUGAAA
UUAAAUUGGCAAAGAAAUUUGACACCUUCAAUGGGGAAUGUCCAAAU
UUUGUAUUUCCCUUAAAUUCCAUAAUCAAGACUAUUCAACCAAGGGU
UGAAAAGAAAAAGCUUGAUGGCUUUAUGGGUAGAAUUCGAUCUGUCU
AUCCAGUUGCGUCACCAAAUGAAUGCAACCAAAUGUGCCUUUCAACU
CUCAUGAAGUGUGAUCAUUGUGGUGAAACUUCAUGGCAGACGGGCGA
UUUUGUUAAAGCCACUUGCGAAUUUUGUGGCACUGAGAAUUUGACUA
AAGAAGGUGCCACUACUUGUGGUUACUUACCCCAAAAUGCUGUUGUU
AAAAUUUAUUGUCCAGCAUGUCACAAUUCAGAAGUAG
(SEQ ID NO: 15)
SARS-COV2 RNA residues 349-394
ACGUGGCUUUGGAGACUCCGUGGAGGAGGUCUUAUCAGAGGCACGU
(SEQ ID NO: 16)
SARS-COV2 RNA residues 564-622
GUGGUGAGACACUUGGUGUCCUUGUCCCUCAUGUGGGCGAAAUACCA
GUGGCUUACCGC
(SEQ ID NO: 17)

FIGS. 6A-6B: Tb-seq on D135 recapitulates previously reported cleavage sites. FIG. 6A: Bar plot showing reactivity profile obtained when probing D135. FIG. 61B: Secondary structure of D135 domain I with Tb³⁺ cleavage sites determined from electrophoresis. The sequence shown in FIG. 6B is also listed in the table below:

D135 ribozyme residues 1-415
(SEQ ID NO: 18)
GAGCGGUCUGAAAGUUAUCAUAAAUAAUAUUUACCAUAUAAUAAU
GGAUAAAUUAUAUUUUUAUCAAUAUAAGUCUAAUUACAAGUGUAU
UAAAAUGGUAACAUAAAUAUGCUAAGCUGUAAUGACAAAAGUAUC
CAUAUUCUUGACAGUUAUUUUAUAUUAUAAAAAAAAGAUGAAGGA
ACUUUGACUGAUCUAAUAUGCUCAACGAAAGUGAAUCAAAUGUUA
UAAAAUUACUUACACCACUAAUUGAAAACCUGUCUGAUAUUCAAU
UAUUAUUUAUUAUUAUAUAAUUAUAUAAUAAUAAAUAAAAUGGUU
GAUGUUAUGUAUUGGAAAUGAGCAUACGAUAAAUCAUAUAACCAU
UAGUAAUAUAAUUUGAGAGCUAAGUUAGAUAUUUACGUAUUUAUG
AUAAAACAGA

FIGS. 7A-7B: Optimizing Tb³⁺ probing conditions. FIG. 7A: Primer extension gel of D135 Tb³⁺ probed for 10 min at the indicated concentrations. The TbCl₃ concentrations are 0, 0.01, 0.1, 0.25, 0.5, 1, and 2 mM. FIG. 7B: Time resolved primer extension gel of D135 probed at 0.5 mM TbCl₃. Times of probing are: 0, 0.1, 0.2 0.5, 1, 2, 3, 5, 7, 10, 20, 30, 45, 60, and 90 min.

FIG. 8: Tb-seq reactivity profile of O.i. intron. Bar plot showing reactivity profile obtained when Tb³⁺ probing O.i. intron at the indicated concentrations.

FIGS. 9A-9B: Tb³⁺ probing the secondary structure of O.i. intron does not result in a distinct cleavage pattern. FIG. 9A: Electrophoretic mobility shift assay of O.i. intron. The MgCl₂ concentrations are 0, 0.1, 0.5, 1, 2, 5, 10, 15, 20, 30 and 50 mM. FIG. 9B: Bar plot displaying reactivity values when probing O.i. intron containing only secondary structure.

FIGS. 10A-10B: Establishing cell lysis Tb³⁺ probing on human RNase P in the presence and absence of proteins. FIG. 10A: Schematic displaying overview of cell lysis probing to either retain or disrupt RNA-protein (RNP) complexes. FIG. 10B: Bar plot displaying Tb³⁻ reactivity values when probed at the indicated concentrations and probing conditions.

FIG. 11: Δ Tb analysis of human RNase P. Secondary structure of RNase P displaying Δ Tb reactivities. For nucleotides in gray, no sequencing data is available. The sequence shown in FIG. 11 is also shown in the table below:

Human RNase P RNA sequence:
(SEQ ID NO: 19)
AUAGGGCGGAGGGAAGCUCAUCAGUGGGGCCACGAGCUGAGUGCG
UCCUGUCACUCCACUCCCAUGUCCCUUGGGAAGGUCUGAGACUAG
GGCCAGAGGCGGCCCUAACAGGGCUCUCCCUGAGCUUCGGGGAGG
UGAGUUCCCAGAGAACGGGGCUCCGCGCGAGGUCAGACUGGGCAG
GAGAUGCCGUGGACCCCGCCCUUCGGGGAGGGGCCCGGCGGAUGC
CUCCUUUGCCGGAGCUUGGAACAGACUCACGGCCAGCGAAGUGAG
UUCAAUGGCUGAGGUGAGGUACCCCGCAGGGGACCUCAUAACCCA
AUUCAGACUACUCUCCUCCGCCCAUU

FIG. 12: Cell lysis probing of SARS-CoV-2 in the presence and absence of proteins. Bar plot displaying reactivity values at the indicated concentrations and probing conditions.

FIG. 13: Δ Tb analysis of 5′ end of SARS-CoV-2. Secondary structure of the terminal 1400 nt of SARS-CoV-2. Secondary structure of the SARS-CoV-2 displaying sites of strong Tb³⁺ cleavage (red). The arrows (black and white) indicate the connectivity of the molecule). Surrounding dots indicate Δ Tb reactivity and are grouped by regions A-Q. The sequence shown in FIG. 13 is SEQ ID NO: 15.

FIGS. 14A-14B: Correlating Tb-seq signal with backbone phosphate distances for Group II O.i. intron. FIG. 14A: Backbone distances (A) for regions displaying Tb³⁺ cleavage. Distances were calculated from phosphate of nucleotide n to phosphate of nucleotide n+2 (Pₙ→Pₙ₊₂). In red text are sites which display strong Tb³⁺ cleavage. Highlighted boxes indicate distances deviating from values in a simple helix in O.i. domain 4. FIG. 14B: Backbone distances (Å) of helix in O.i. domain 4. Distances were calculated from phosphate of nucleotide n to phosphate of nucleotide n+2 (Pₙ→Pₙ₊₂). All values calculated from PDB 4E8M.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

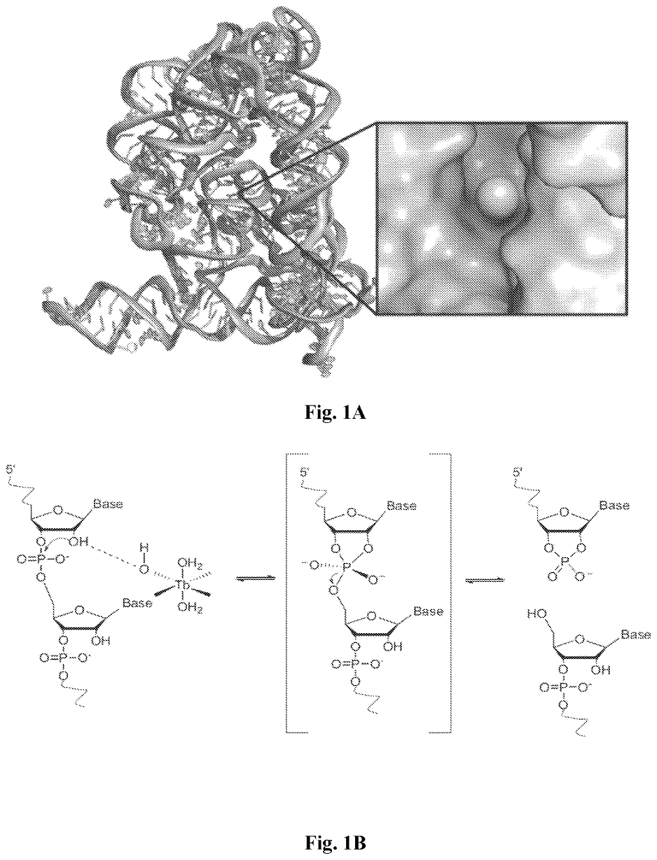

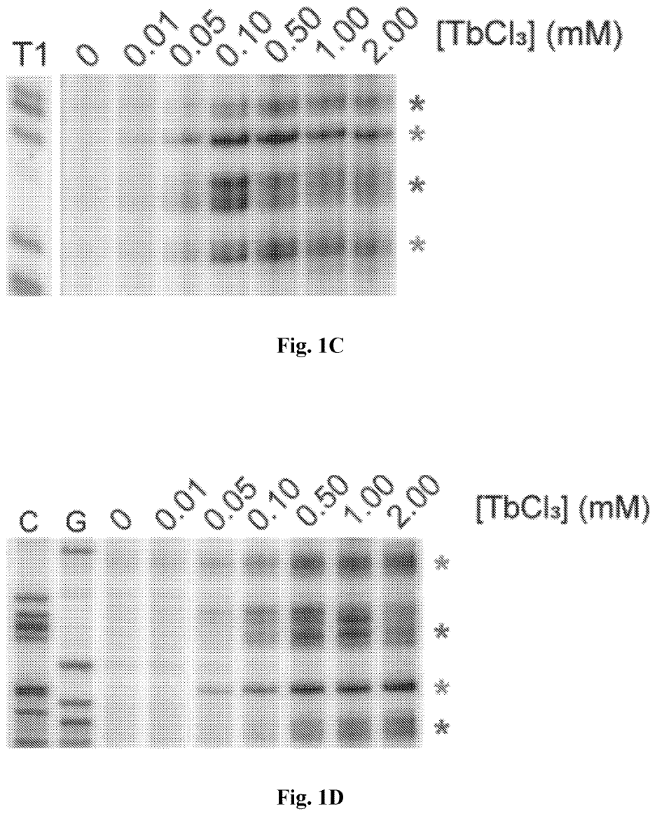

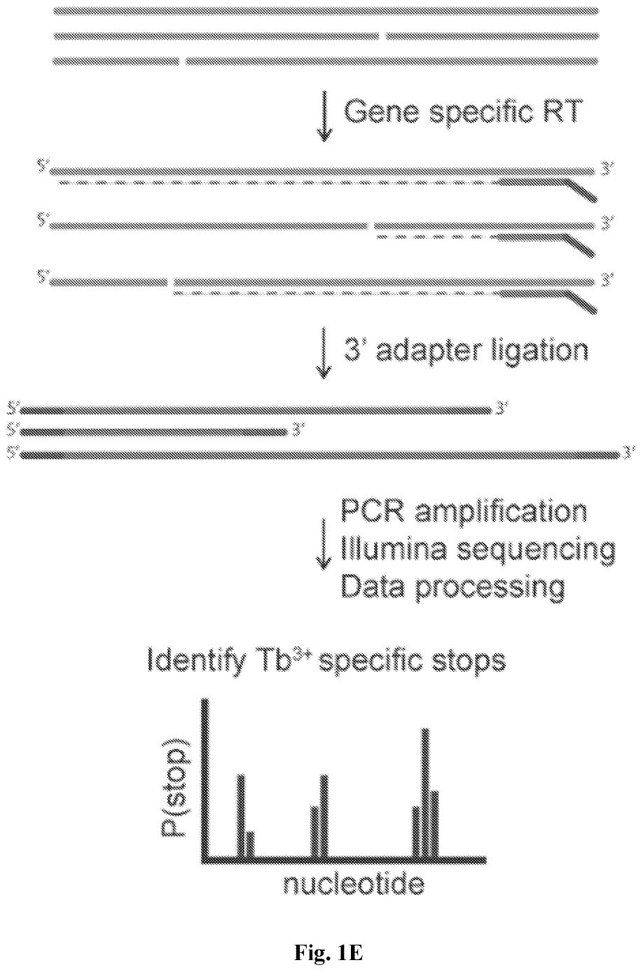

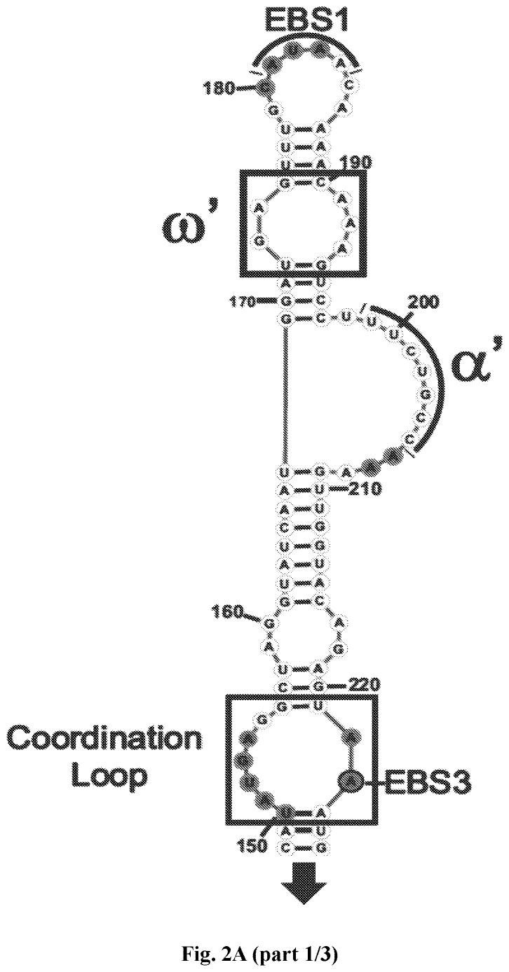

High-resolution RNA structures show that regions of tightly packed tertiary structure often contain phosphate backbones that are packed in close proximity, within the same strand or on adjacent strands. These local regions of intense negative electrostatic potential act as sinks for multivalent ion coordination (FIG. 1A). One non-limiting way to probe these electrostatically negative reservoirs is to monitor the cleavage patterns catalyzed by coordinated metal ions. When nucleotides in such regions adopt an “in-line geometry”, which aligns an upstream 2′-OH with the downstream 3′-OR group of a phosphodiester linkage, adjacent metal hydroxide ions can behave as a general base, deprotonating the 2′-OH group and producing a 2′ oxyanion nucleophile that attacks the adjacent phosphate and causes strand scission.

While this type of Mg²⁺-catalyzed cleavage (known as “in-line probing”) normally occurs on a slow timescale that ranges from hours to days, the same phenomenon is greatly accelerated by lanthanide ions such as terbium (Tb³⁺) and europium (Eu³⁺). Tb³⁺ and Mg²⁺ share similar ionic radii and coordination geometry preferences, but lanthanide ions have an additional positive charge and the pKa of coordinated water molecules is much lower for ions such as Tb³⁺ (pKa ~7.9 for Tb³⁺—H₂O versus ~11.4 for Mg²⁺—H₂O). Therefore, at low ion concentrations and neutral pH. Tb³⁺ coordinates with structured RNA binding sites in a manner that is almost identical to that of Mg²⁺, but the more potent Tb³⁺ general base rapidly facilitates RNA backbone cleavage at sites of metal ion binding (FIG. 1B). Tb³⁺ probing of RNA has been used extensively in the past, but until now, it was a low-throughput method that relied on electrophoretic quantification.

The present disclosure relates, in one aspect, to Tb-seq, a sequencing-based approach that employs Tb³⁺ to detect regions of tertiary structure in long RNAs. To demonstrate the efficacy of this technique, the Tb-seq pipeline was first applied to identify tertiary structural motifs within structurally well-characterized RNA molecules. The Tb-seq pipeline was then applied to probe known and unknown RNA structures in a cellular context to investigate RNA motifs and protein binding sites. These studies show that Tb-seq detects regions of RNA involved in RNA tertiary structure motifs and within RNP complexes, thereby providing a powerful new approach for pinpointing regions of complex RNA structure that are potentially associated with RNA functional elements.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, NY, and Ausubel et al., 2002. Current Protocols in Molecular Biology, John Wiley & Sons. NY), which are provided throughout this document.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The articles “a” and “an” are used herein to refer to one or to more than one (i.e., to at least one) of the grammatical object of the article. By way of example. “an element” means one element or more than one element. The term “or” is used to refer to a nonexclusive “or” unless otherwise indicated. The statement “at least one of A and B” or “at least one of A or B” has the same meaning as “A, B, or A and B.”

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“About” as used herein when referring to a measurable value such as an amount, a temporal duration, and the like, is meant to encompass variations of ±20% or ±10%, in certain embodiments ±5%, in certain embodiments ±10%, in certain embodiments ±0.1% from the specified value, as such variations are appropriate to perform the disclosed methods.

A disease or disorder is “alleviated” if the severity of a sign or symptom of the disease or disorder, the frequency with which such a sign or symptom is experienced by a patient, or both, is reduced.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also comprises the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

As used herein, “conjugated” refers to covalent attachment of one molecule to a second molecule.

The term “diagnosis,” as used herein, refers to the determination of a condition of a subject, such as determining whether the subject has a particularly disease condition, susceptibility, or other trait.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “DNA” as used herein is defined as deoxyribonucleic acid.

An “effective amount” or “therapeutically effective amount” of a compound is that amount of a compound which is sufficient to provide a beneficial effect to the subject to which the compound is administered.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

As used herein, the term “handle” refers to a functional component an oligonucleotide sequence which itself is an oligonucleotide or polynucleotide sequence that provides an annealing site for amplification of the construct oligonucleotide sequence. Handles used in the present embodiments can be formed of polymers of DNA, RNA, PNA, modified bases or combinations of these bases, or polyamides, and so forth. In some embodiments, the universal handles used in the embodiments disclosed herein are about 10 of such monomeric components, e.g., nucleotide bases, in length. In other embodiments, the handle is at least about 5 to 160 monomeric components, e.g., nucleotides, in length. Thus in various embodiments, the handles described herein are at least 5, 6, 7, 8, 9, 10, 11, 12, 13, 14, 15, 16, 17, 18, 19, 20, 21, 22, 23, 24, 25, 26, 27, 28, 29, 30.31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146,147, 148, 149, 150, 151, 152, 153, 154, 155, 156, 157, 158, 159, 160, or more than 160 monomeric components, e.g., nucleic acids. As such, the handles described herein can be generic sequences suitable as an annealing site for extension by a polymerase as described elsewhere herein, e.g., a reverse transcriptase, a DNA polymerase, or the like, or contain extension primer binding sites, sequencing primer binding sites, or the like.

The term “homology” refers to a degree of complementarity. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. “Hybridize” as used herein refers to two full complementary or partially complementary single-stranded DNA or RNA molecules form a single double-stranded molecule through base pairing.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide contains at a minimum, the sense or coding strand (i.e., the oligonucleotide may be single-stranded), but may contain both the sense and anti-sense strands (i.e., the oligonucleotide may be double-stranded).

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides (e.g., proteins and enzymes) are found in the state they exist in nature.

The term “next generation sequencing”, “NGS”, “massive parallel sequencing”, “massively parallel sequencing”, or “second-generation sequencing” refers to any number of massive parallel sequencing that use massive parallel sequencing via spatially separated, clonally amplified DNA templates or single DNA molecules in a flow cell. DNA sequencing libraries are first generated by clonal amplification by PCR in vitro. Then, the DNA is sequenced by synthesis, such that the DNA sequence is determined by the addition of nucleotides to the complementary strand rather than through chain-termination chemistry. Then, the spatially segregated, amplified DNA templates are sequenced simultaneously in a massively parallel fashion without the requirement for a physical separation step. These steps are followed in most NGS platforms, but with distinct strategies. Non-limiting examples of NGS platforms include Roche 454, GS FLX Titanium, Illumina MiSeq, Illumina NextSeq. Illumina HiSeq, Illumina Genome Analyzer IIX, Life Technologies SOLiD4. Life Technologies Ion Proton, Complete Genomics, Helicos Biosciences Heliscope, and Pacific Biosciences SMRT.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “3′” refers to a region or position in a single-stranded polynucleotide or oligonucleotide that is downstream from another region or position in the same polynucleotide or oligonucleotide. The term “3′ end” refers to the 3′ terminus of the polynucleotide containing a 3′ phosphate group.

The term “5′” refers to a region or position in a polynucleotide or oligonucleotide upstream from another region or position in the same polynucleotide or oligonucleotide. The term “5′ end” refers to the 5′ terminus of the polynucleotide containing a 5′ phosphate group.

The term “oligonucleotide” as used herein can include single- and double-stranded DNA (ssDNA and dsDNA), single- and double-stranded RNA (ssRNA and dsRNA), chemically modified polynucleotides or polynucleosides, and combinations thereof. Polynucleotides are polymers of nucleotides joined through phosphodiester or phosphorothioate linkages. A nucleoside consists of an adenine (A), guanine (G), cytosine (C), thymine (T), or uracil (U) bonded to a sugar moiety. A nucleotide is a phosphate ester of a nucleoside. The nucleoside units in DNA contain deoxyribose sugars.

The terms “patient,” “subject,” “individual,” and the like are used interchangeably herein, and refer to any animal, or cells thereof whether in vitro or in vivo, amenable to the methods described herein. In some non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

The term “recombinant polypeptide” as used herein is defined as a polypeptide produced by using recombinant DNA methods.

“Reverse transcriptase” or “RT” as used herein is an enzyme that is capable of synthesizing complementary DNA (cDNA) strands from RNA templates. A RT can be composed of distinct domains, having activities such as RNA-dependent DNA polymerase activity and RNase H activity, and/or DNA-dependent DNA polymerase activity. In the presence of an annealed primer, the RT binds to an RNA template and initiates the reaction. RNA-dependent DNA polymerase activity synthesizes the complementary DNA (cDNA) strand, incorporating dNTPs. RNase H activity degrades the RNA template of the DNA:RNA complex. DNA-dependent DNA polymerase activity (if present) recognizes the single-stranded cDNA as a template, uses an RNA fragment as a primer, and synthesizes the second-strand cDNA. Double-stranded cDNA is thus formed. The processivity of a RT refers to the number of nucleotides incorporated in a single binding event of the enzyme. Therefore, a highly processive reverse transcriptase can synthesize longer cDNA strands in a shorter reaction time. Some engineered MMLV (Moloney Murine Leukemia Virus) reverse transcriptases can add as many as 1,500 nucleotides in a single binding event, which represents a processivity that is about 65 times greater than that of wild-type MMLV reverse transcriptase. A non-limiting example of a reverse transcriptase is MarathonRT.

The term “RNA” as used herein is defined as ribonucleic acid.

A “therapeutic” treatment is a treatment administered to a subject who exhibits signs or symptoms of a disease or disorder, for the purpose of diminishing or eliminating those signs or symptoms.

As used herein. “treating a disease or disorder” means reducing the severity and/or frequency with which a sign or symptom of the disease or disorder is experienced by a patient.

The term “substantially reduce” as used herein refers to reducing the amount of a majority of, or mostly, a substance, by at least about 5%, 10%, 15%, 20%, 30%, 40%, 50%, 60%, 70%, 80%, 90%, 95%, 96%, 97%, 98%, 99%. 99.5%. 99.9%. 99.99%, or at least about 99.999% or more, or 100%, relative to an initial amount of the substance.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, a peptide, or a nucleic acid (e.g., RNA or DNA) with a second chemical species, to mean that the interaction is dependent upon the presence of a particular sequence or structure (e.g., a specific nucleotide sequence or epitope) on the chemical species; for example, an siRNA or riboswitch recognizes and binds to a specific nucleotide sequence rather than to nucleic acid molecules generally. If a siRNA is specific for sequence “A”, the siRNA will specifically bind to a molecule containing sequence A.

Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example. 1, 2, 2.7, 3, 4, 5, 5.3, and 6. This applies regardless of the breadth of the range.

### Methods

In some aspects, the present invention is directed to a method of probing tertiary structure of a ribonucleotide (RNA) molecule.

In some embodiments, the method includes contacting the RNA molecule with terbium (III) (Tb³⁺) under conditions that allow for Tb³⁺-mediated cleavage of the RNA molecule, thus generating at least one RNA fragment.

In some embodiments, the method further includes determining at least a fraction of the sequence of the at least one RNA fragment.

In some embodiments, the method further includes identifying at least a portion of the tertiary structure in the RNA molecule based on the identified Tb³⁺-mediated cleavage site.

In some embodiments, the method further includes identifying at least one site of the Tb³⁺-mediated cleavage based on the determined sequence of the at least one RNA fragment.

In contrast to conventional methods, which can only locate tertiary structures in RNA molecules of smaller sizes, the method herein is able to probe tertiary structure in RNA molecules of virtually all sizes. In some embodiments, the RNA molecule includes at least 2-100.000 nucleotides. In some embodiments, the RNA molecule includes 10 or more nucleotides, such as 20 or more nucleotides, 50 or more nucleotides, 100 or more nucleotides, 200 or more nucleotides, 500 or more nucleotides, 1,000 or more nucleotides, 2,000 or more nucleotides, 5,000 or more nucleotides, 10,000 or more nucleotides, 20,000 or more nucleotides, or 50,000 or more nucleotides.

### Tb³⁺-Mediated Cleavage of the RNA Molecule

Tb³⁺-mediated cleavage of the RNA molecule depends on various conditions such as the nature of the RNA molecule (e.g., size, sequence, structure, etc), the concentration of the RNA molecule, the purity of the RNA molecule (e.g., purified vs a mixture of cellular components such as RNA/proteins/lipids, such as in a cell lysate), the temperature, the concentrations of the Tb³⁺, the type and concentrations of other ions present in the system, the reaction time, and the like. In some embodiments, except for the nature of the RNA molecule, other factors, especially the concentration of Tb³⁺, can be varied to identify suitable and/or desirable conditions.

In some embodiments, the conditions are controlled such that non-structural-specific Tb³⁺-mediated cleavage of the RNA molecule are eliminated, reduced and/or minimized, and structural-specific Tb³⁺-mediated cleavage of the RNA molecule are enhanced and/or maximized.

In some embodiments, the site of the Tb³⁺-mediated cleavage is within or adjacent to the tertiary structure. Since structural-specific Tb³⁺-mediated cleavages often take place within or adjacent to tertiary structures of RNA molecules, identifying the location of these Tb³⁺-mediated cleavages allows tertiary structures to be identified and/or located.

### Sequencing and Sample Preparation Therefor

Methods for sequencing the at least one RNA fragment generated from the Tb³⁺-mediated cleavage are not limited within the methods of the disclosure, and any methods of directly or indirectly sequencing RNA molecules can be used therein.

In some embodiments, at least a portion of the sequence of the at least one RNA fragment is determined by capillary electrophoresis or by a high-throughput method such as but not limited to a next generation sequencing (NGS) method.

In some embodiments, the sequence of the at least one RNA fragment is determined using a NGS method. Such methods allow the sequencing of RNA molecules with different sequences and/or different sizes: they can be employed, for example, when the size of the RNA molecule is large, the RNA molecule has a complex tertiary structure, and/or several RNA molecules are to be probed simultaneously. Furthermore, the NGS sequencing methods do not require the purification of the RNA fragments (or cDNA molecules thereof). Next generation sequencing is described in, for example, Goodwin et al. (Nature Reviews Genetics 17, 333-351 (2016)), Hu et al. (Human Immunology 82, Issue 11, 801-811 (2021)), McCombie et al. (Cold Spring Harb Perspect Med 2019; 9:a036798) and Kumar et al. (Semin Thromb Hemost 2019; 45(07): 661-673).

Furthermore, the at least one RNA fragment can be sequenced directly as RNA fragment(s) or the at least one RNA fragment can be to DNA before being sequenced.

In some embodiments, the at least one RNA fragment is sequenced directly. In some embodiments, the at least one RNA fragment is converted to a DNA molecule and then sequenced. Method of sequencing RNA directly or indirectly includes Pacific Biosciences (PacBio) single-molecule real-time (SMRT) sequencing, Helicos single-molecule fluorescent sequencing, Oxford Nanopore Technologies (ONT) nanopore sequencing, any Illumina technologies (e.g., TruSeq), and the like.

In some embodiments, the at least one RNA fragment is converted to a DNA molecule by a reverse transcriptase (RT) and then sequenced. The choice of the reverse transcriptase is not limited within the present disclosure. In some embodiments, the RT includes a group II intron RT (see e.g., Mohr et al., RNA. 2013 July; 19(7):958-70), a retroviral RT, and the like. Non-limiting examples of RTs include a superscript series RT, a Moloney Murine Leukemia Virus RT, MarathonRT, TGIRT, and the like. Since the ability for an RT to convert long RNA fragments into cDNA molecule allows long RNA molecules to be probed, in some embodiments, the RT is a high processive RT (i.e., the RT is able to process long stretches of RNA chains into cDNA before the reverse transcription stops). In some embodiments, the RT has a processivity of about 500 nucleotides or longer, such as 1,000 nucleotides or longer, 2,000 nucleotides or longer, 5,000 nucleotides or longer, 10,000 nucleotides or longer, or 20.000 nucleotides or longer.

In some embodiments, the at least one RNA fragment is processed into a cDNA library before being sequenced. In some embodiments, the DNA molecules in the cDNA library are cloned into a vector or attached with an identification sequence, such as a barcoding sequence. Building cDNA library, such as stop-based RT library, is described in, for example, Strobel et al. (Nat Rev Genet. 2018 October; 19(10): 615-634).

In some embodiments, the at least one RNA fragment or the cDNA molecules in the cDNA library are attached with a barcoding sequence. In the case of adding barcoding sequences to the RNA fragment, direct ligation can be used. In the case of adding barcoding sequences to the cDNA molecule, the barcoding sequence can be attached either at the reverse transcription step, or during the subsequent amplification steps. According to some embodiments, for the RNA fragment and linear cDNA molecules, the barcoding sequence can be added at the 5′-end, the 3′-end, or both. According to some embodiments, for circular cDNA molecules, the barcoding sequence can be included in the portion linking the sequences corresponding to the 5′-end and the 3′-end of the RNA fragment.

In some embodiments, the reverse transcription is performed in the present of a gene-specific RT primer containing a 5′-adapter handle. In some embodiments, the gene-specific RT primer comprises a 5-adapter handle. In some embodiments, the resulting cDNA molecule(s) comprise(s) a 3′-adapter handle. In some embodiments, the cDNA molecules(s) comprising a 3′-adapter handle is/are amplified using a polymerase-chain reaction (PCR). In some embodiments, the PCR reaction is performed using multiplex handles.

### Sequencing Data Processing

The sequencing data processing sometimes depends on the choice of the sequencing methods, as well as the method of sample preparation for sequencing. In some embodiments, the sequence data processing increases the accuracy and reliability of the identification Tb³⁺ -mediated cleavage, especially the identification of the tertiary structure-dependent Tb³⁺-mediated cleavage.

In some embodiments, the sequence of the cDNA molecules obtained in the sequencing step is aligned with the sequence of the RNA molecule.

In some embodiments, a stop probability of one or more nucleotide (i.e., the probability of a nucleotide appears at an end of the RNA fragment—i.e., the site where the RNA fragment “stops”) in the RNA molecule is calculated. In some embodiments, the calculated stop probability of the one or more nucleotide is compared to a predetermined value. In some embodiments, a stop probability equal to or higher than the predetermined value indicates that the nucleotide is at the site of the Tb³⁺-mediated cleavage. Since not all “stops” are caused by Tb³⁺-mediated cleavages, and sometimes the Tb³⁺-mediated cleavages are low-frequency non-tertiary structure-associated cleavages, the removal of low stop probability nucleotides increases the accuracy and reliability of the Tb³⁺-mediated cleavage site identification.

The method of calculating the stop probability is not limited within the present disclosure. Examples of stop probability calculation methods are described in, for example, Loughrey et al. (Nucleic Acids Res. 2014 Dec. 1: 42(21): e165). In some embodiments, the stop probability per RNA molecule nucleotide is calculated as an abundance of stops for the RNA molecule nucleotide in relative to a sum of the total number of read-through events plus the number of stops for the RNA molecule nucleotide.

In some embodiments, the stop probability per RNA molecule nucleotide in the absence of Tb³⁺ probing/cleaving is subtracted from the stop probability per RNA molecule nucleotide, thus providing an RNA molecule nucleotide reactivity value. In some embodiments, the stop probability per RNA molecule nucleotide in the absence of Tb³⁺ contacting is obtained in the same manner, except for that the RNA molecule is not contacted with Tb³⁺. In certain embodiments, this allows the reduction or elimination of background noise caused by non-Tb³⁺ induced cleavage.

In some embodiments, only the RNA molecule nucleotides that have read-throughs equal to or greater than a predetermined number are further considered. In some embodiments, the predetermined number is 100, 200, 500, 1,000, 2,000, 5.000, 10.000, 20,000, 50,000, or 100,000, or any multiple, fraction, and/or combination thereof.

In some embodiments, the RNA molecule nucleotide reactivity value is normalized to a predetermined value of top percentile of the stop rates for the RNA molecule. In some embodiments, the top percentile is about top 40%, about top 25%, about top 20%, about top 15%, about top 10%, or about top 5%.

In some embodiments, the normalized RNA molecule nucleotide reactivity value is scaled at a predetermined value. The scaling of the normalized RNA molecule nucleotide reactivity value is not limited within the present disclosure. In some embodiments, the scale is 0-2, 1-8, 0-10, or any scale considered to be relevant to the specific sequencing method.

In some embodiments, only the RNA molecule nucleotides with normalized reactivity values equal to or higher than a predetermined value are further considered as potential site-bound Tb³⁺-dependent cleavage-sites.

In some embodiments, the RNA molecule is contacted with two or more distinct Tb³⁺ concentrations, and the identified tertiary structure in the RNA molecule is based on one or more identified Tb³⁺-mediated cleavage sites that show dependence on Tb³⁺ concentrations.

### Identifying Tertiary Structure Induced or Disrupted by Binding Partners of RNA Molecule

Tertiary structures in an RNA molecule are sometimes induced or disrupted by binding partners of the RNA molecule (which include other RNA molecules, DNA molecules and proteins). In some embodiments, the methods herein identify tertiary structures induced or disrupted by the binding partner of the RNA molecule.

In some embodiments, the method is performed separately with the RNA molecule free of the binding partner, and the RNA molecule in the presence of the binding partner.

In some embodiments, the binding partner is another RNA molecule, a DNA molecule, a protein, or combinations thereof.

In some embodiments, the RNA molecule being probed is a purified RNA molecule, and the binding partner is contacted with the purified RNA molecule so that the method can be performed in the present of the binding partner.

In some embodiments, the RNA molecule being probed is already in complex with the binding partners (for example, in a cell or a cell lysate), and the RNA molecule free of the binding partner needs to be generated by separating the binding partner from the RNA molecule. In some embodiments, the DNA binding partner is separated/removed by an enzyme cleaving DNA molecules, such as a DNase. In some embodiments, the protein binding partner is separated/removed by cleaving the protein with a proteolytic enzyme, such as a protease.

In some embodiments, the method identifies a tertiary structure induced by the binding partner, or a tertiary structure disrupted by the binding partner in the RNA molecule, based on a change in a pattern of the Tb³⁺-mediated cleavage.

### Kits

In some aspects, the present invention is directed to a kit for probing tertiary structures in an RNA molecule.

In some embodiments, the kit includes Tb³⁺; a buffer solution (or concentrates or powder forms thereof) for dissolving the RNA molecule and allowing the Tb³⁺ to cleave the RNA molecule in a tertiary structure dependent manner; and a manual instructing that the RNA molecule is to be cleaved in the buffer solution in the presence of the Tb³⁺.

In some embodiments the components included in the kit are provided in the form of a concentrate or a powder form. In some embodiments, the concentrate or a powder simplifies the preparation of a system for the Tb³⁺-mediated cleavage of the RNA molecule, as the RNA molecule sometimes exists in a sample that have buffer components, ions and other components different from those suitable for carrying out the Tb³⁺-mediated cleavage of the RNA molecule, and the components in the concentrate or powder form makes adjusting these parameters more convenient.

In some embodiments, the Tb³⁺ is dissolved in the buffer solution. In some embodiments, the Tb³⁺ is provided as a Tb (III) salt, such as TbCl₃. In some embodiments, the manual instructs that the Tb (III) salt, such as the TbCl₃, is to be dissolved in the buffer solution. In some embodiments, the concentration of the Tb³⁺ in the buffer solution ranges from about 0.01 mM to about 200 mM, such as from about 0.02 mM to about 100 mM, from about 0.05 mM to about 50 mM, from about 0.1 mM to about 20 mM, or from about 0.2 mM to about 10 mM. In some embodiments, the concentration of the Tb³⁺ in the buffer solution is about 0.01 mM, about 0.02 mM, about 0.05 mM, about 0.1 mM, about 0.2 mM, about 0.5 mM, about 1 mM, about 2 mM, about 5 mM, about 10 mM, about 20 mM, about 50 mM, or any ranges therebetween. In some embodiments, the manual instructs that the Tb (III) salt is to be dissolved to yield the above concentrations.

The choice of the buffer solution (or the concentrates/powder thereof) are not limited and most, if not all, Good's buffers (Good et al., Biochemistry. 1966 February; 5(2):467-77) can be used. Non-limiting examples of the buffers include ACES buffer, ADA buffer, AMP buffer, AMPD buffer. AMPSO buffer, BES buffer. Bicine buffer, Bis-Tris buffer, Bis-6Tris Propane buffer, CABS buffer, CAPS buffer, CAPSO buffer, CHES buffer, DIPSO buffer, EPPS (HEPPS) buffer, Gly-Gly buffer, HEPBS buffer, HEPES buffer, HEPPSO buffer, MES buffer, MOBS buffer, MOPS buffer, MOPSO buffer, PIPES buffer, POPSO buffer, TABS buffer, TAPS buffer, TAPSO buffer, TES buffer, Tricine buffer, and the like.

In some embodiments, the concentration of a component of the buffer solution ranges from about 0.01 mM to about 200 mM, such as from about 0.02 mM to about 100 mM, from about 0.05 mM to about 50 mM, from about 0.1 mM to about 20 mM, or from about 0.2 mM to about 10 mM. In some embodiments, the concentration of a component of the buffer solution is about 0.01 mM, about 0.02 mM, about 0.05 mM, about 0.1 mM, about 0.2 mM, about 0.5 mM, about 1 mM, about 2 mM, about 5 mM, about 10 mM, about 20 mM, about 50 mM, or any ranges therebetween. In some embodiments, the manual instructs that the buffer concentrate is to be diluted to the above concentrations, or that the buffer powder is to be dissolved to the above concentrations.

In some embodiments, the kit further includes a monovalent salt, a divalent salt, or combinations thereof. Non-limiting examples of the monovalent salt includes a sodium salt (such as NaCl), a potassium salt (such as KCl), an ammonium salt (such as ammonium sulfate), and the like. Non-limiting examples of the divalent salt include a magnesium salt (such as MgCl₂), a calcium salt (such as CaCl₂)), a manganese salt (such as MnCl₂), and the like.

In some embodiments, the monovalent salt and/or the divalent salt is dissolved in the buffer solution. In some embodiments, the monovalent salt and/or the divalent salt is provided as a concentrate or in powder form. In some embodiments, the concentration of the monovalent salt and/or the divalent salt in the buffer solution is about 0.01 mM, about 0.02 mM, about 0.05 mM, about 0.1 mM, about 0.2 mM, about 0.5 mM, about 1 mM, about 2 mM, about 5 mM, about 10 mM, about 20 mM, about 50 mM, about 100 mM, about 200 mM, about 500 mM, about 1 M. or any ranges there between. In some embodiments, the manual instructs that the concentrate of the monovalent salt and/or the divalent salt is to be diluted to the above concentrations, or that the powder of the monovalent salt and/or the divalent salt is to be dissolved to the above concentrations.

In some embodiments, the kit further includes an RNase inhibitor. The RNase inhibitor allows the RNA molecule whose tertiary structures to be probed to be more stable in the buffer solution, and can reduce the non-Tb³⁺ related cleavage of the RNA molecule. Non-limiting examples of RNase inhibitors include SUPERase-In. In some embodiments, the RNase inhibitor is provided as a concentrate, a powder, or pre-dissolved in the buffer solution.

In some embodiments, the kit further includes a component for preparing the sample for sequencing, after the Tb³⁺ mediated cleavage reaction is complete. Non-limiting examples of components for preparing the sample for sequencing includes a barcoding nucleic acid (for e.g., incorporating a barcoding sequence), a ligase (for e.g., ligating a barcoding sequence or incorporating an adaptor for circular consensus sequencing), a primer (for e.g., amplifying cDNA reverse transcribed from RNA fragments or generating cDNA from RNA), a reverse transcriptase (for e.g., reverse transcription), a DNA polymerase (for e.g., amplifying cDNA reverse transcribed from RNA fragments), an RNase (for e.g., removing residual RNA fragments in the case of cDNA based sequencings), and the like. In some embodiments, the manual further instructs that the samples obtained from Tb³⁺ mediated RNA cleavage is to be prepared for sequencing, such as a next generation sequencing.

In some embodiments, the kit is for probing tertiary structures of an RNA molecule in a cell, and the cell needs to be lysed so that the RNA molecule can be released and contact the Tb³⁺.

In some embodiments, the kit further includes a detergent for lysing the cell. Non-limiting examples of detergents suitable for lysing cells include TritonX-100, IGEPAL, SDS, NP-40, and the like. In some embodiments, the detergent is provided as substantially pure form, as concentrates, or dissolved into the buffer solution.

In some embodiments, the kit allows the study of the RNA molecule in complex with a protein binding partner. In some embodiments, the kit further includes a protease inhibitor such that the protein binding partner are not destroyed by proteases, such as proteases presented in cell lysates.

In some embodiments, the kit allows the study of the RNA molecule, which is in complex with a protein binding partner in a cell or a cell lysate, in free form. In some embodiments, the kit further includes a proteolytic enzyme for cleaving the protein binding partner. Non-limiting examples of the proteolytic enzyme include Proteinase-K.

In some embodiments, the protease inhibitor or the proteolytic enzyme is provided as a concentrate, as substantially pure form, or dissolved in the buffer solution.

In some embodiments, the kit further includes a control RNA molecule. In some embodiments, the condition by which the Tb³⁻-mediated cleavage of the control RNA molecule, and/or the patterns of the sequencing results of the RNA fragment(s) generated from the Tb³⁺-mediated cleavage of the control RNA molecule is known, such that the Tb³⁺-mediated cleavage of the control RNA molecule and the patterns of the sequencing results can be used to ensure that the components of the kits are in working condition and/or that the experiment procedures are correctly carried out by the practitioners.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Systematic Detection of Tertiary Structural Modules in Large RNAs and RNP Interfaces by Tb-Seq

Compact RNA structural motifs control many aspects of gene expression, but the art lacks methods for finding these structures in the vast expanse of multi-kilobase RNAs. To adopt specific 3-D shapes, many RNA modules must compress their RNA backbones together, bringing negatively charged phosphates into close proximity. This is often accomplished by recruiting multivalent cations (usually Mg²⁺), which stabilize these sites and neutralize regions of local negative charge. Coordinated lanthanide ions, such as terbium (III) (Tb³⁺), can substitute for Mg²⁺ at these sites, where they induce efficient RNA cleavage, thereby revealing compact RNA 3-D modules. Until now, Tb³⁺ cleavage sites were monitored via low-throughput biochemical methods only applicable to small RNAs. Here the present study presents Tb-seq, a high-throughput sequencing method for detecting compact tertiary structures in large RNAs. Tb-seq detects sharp backbone turns found in RNA tertiary structures and RNP interfaces, providing a way to scan transcriptomes for stable structural modules and potential riboregulatory motifs.

RNAs can adopt complex folded motifs and higher-order 3-D structures that are essential across a variety of specific cellular processes. It has recently become clear that many types of multi-kilobase RNA transcripts contain regions of tertiary structure that, either alone or in concert with protein partners, carry out biological function. However, identifying these regions of complex RNA structure remains challenging. Current structure prediction methods on long RNAs are unable to pinpoint regions containing stable RNA tertiary structure modules or complex protein binding sites from sequence alone. While biophysical techniques such as NMR, x-ray crystallography and cryo-EM are invaluable tools for the observation of RNA structure, they are time consuming and difficult to perform on a multi kilobase-length RNA that contains a mixture of both structured and flexible regions. As the understanding of their biological functions becomes increasingly important, and interest in small molecule targeting of RNAs grows, it is vital to develop new tools for identifying regions of tertiary structure in long RNA molecules.

In recent years, chemical probing has become a powerful tool for studying RNA structure. Many important advances have improved our ability to identify single-versus double-stranded nucleotides in RNA, and these data have primarily been used to infer secondary but not tertiary structures of RNA. Fewer methods have been developed to detect higher order structure and these protocols are limited to an assessment of solvent accessible regions or identification of long-range RNA-RNA base-pairs by cross-linking methods or statistical correlations in mutational profiling. The field would benefit from a readily adaptable, high throughput approach for identifying regions of local tertiary structure, which are often hallmarks of functional RNA motifs and riboregulatory elements.

High-resolution RNA structures show that regions of tightly packed tertiary structure often contain phosphate backbones that are packed in close proximity, within the same strand or on adjacent strands. These local regions of intense negative electrostatic potential act as sinks for multivalent ion coordination (FIG. 1A). One way to probe these electrostatically negative reservoirs is to monitor the cleavage patterns catalyzed by coordinated metal ions. When nucleotides in such regions adopt an “in-line geometry”, which aligns an upstream 2′-OH with the downstream 3′-OR group of a phosphodiester linkage, adjacent metal hydroxide ions can behave as a general base, deprotonating the 2′-OH group and producing a 2′ oxyanion nucleophile that attacks the adjacent phosphate and causes strand scission. While this type of Mg²⁺-catalyzed cleavage (known as in-line probing) normally occurs on a slow timescale that ranges from hours to days, the same phenomenon is greatly accelerated by lanthanide ions such as terbium (Tb³⁺) and europium (Eu³). Tb³⁺ and Mg²⁻ share similar ionic radii (0.92 Å and 0.72 Å, respectively) and coordination geometry preferences for oxygen, but lanthanide ions have an additional positive charge and the pKa of coordinated water molecules is much lower for ions such as Tb³⁺, (pKa ~7.9 for Tb³⁺—H₂O versus ~11.4 for Mg²⁺—H₂O). Therefore, at low ion concentrations and neutral pH, Tb³⁺ coordinates with structured RNA binding sites in a manner that is similar to that of Mg²⁺, but the more potent Tb³⁺ general base rapidly facilitates RNA backbone cleavage at sites of metal ion binding³¹ (FIG. 1B).

Here the present study present Tb-seq, a sequencing-based approach that employs Tb³⁺ to detect regions of tertiary structure in long RNAs. To demonstrate the efficacy of this technique, the present study first applied the Tb-seq pipeline to identify tertiary structural motifs within structurally well-characterized RNA molecules. The present study then applied it to probe known and unknown RNA structures in a cellular context to investigate RNA motifs and protein binding sites. These studies show that Tb-seq detects regions of RNA involved in RNA tertiary structure motifs and within RNP complexes, thereby providing a powerful new approach for pinpointing regions of complex RNA structure that are potentially associated with RNA functional elements.

### Example 2: Materials and Methods

### In Vitro Transcription and Purification

The in vitro transcription of aI5γ D135 and Oceanobacillus iheyensis (O.i.) group II intron D1-5, was performed as previously described (Qin & Pyle, 1997, Biochemistry 36, 4718-4730). In brief, RNAs were transcribed by runoff transcription using T7 RNA polymerase in a buffer containing 12 mM MgCl₂, 40 mM Tris-Cl pH8, 2 mM Spermidine, 10 mM NaCl. 0.01% Triton X-100, 10 mM DTT. 5 μl SUPERase-In and 3.6 mM of each NTP. The reactions were incubated at 37° C. for 2 hours followed by purification on a denaturing 5% polyacrylamide gel. The in vitro transcription of the full-length HCV genome (JCI) was performed as previously described (Wan, et al., 2022. J. Virol. 96, e0194621-e0194621). Transcribed RNA was buffer exchanged into a filtration buffer (50 mM HEPES pH7.2 and 150 mM KCl) using 50-kDa Amicon Ultra filtration columns. The RNA was purified by size exclusion chromatography at room temperature using a self-packed 24 ml Sephacryl S-1000 column equilibrated with filtration buffer. RNA from the peak fraction was used for subsequent folding and probing.

### RNA Folding and Tb³⁺ Probing

For D135, Tb³⁺ cleavage was performed using two approaches. The first was direct visualization of Tb³⁺ mediated RNA cleavage by electrophoresis. In brief, D135 was 5′ end-labeled with [γ-³²P] ATP using T4 polynucleotide kinase. Thereafter, 3 nM of ³²P-labeled RNA and 1 μg of unlabeled RNA were mixed in a monovalent buffer containing 50 mM MOPS pH7 and 500 mM KCl to a final volume of 18 μl. For visualization of Tb³⁺ mediated cleavage sites by reverse transcription and sequencing, 1 μg of unlabeled RNA was used. For all reactions, the mixture was heated up to 90° C. for 1 min and cooled at room temperature for 2 min. Thereafter, 2 μl of 1M MgCl₂ (final concentration 100 mM) was added and folded at 37° C. for 30 min. Subsequently, probing was performed by incubating 18 μl of the folded RNA with 10×TbCl₃ stocks prepared in the monovalent buffer (final 1× concentration from 0.01 mM-2 mM TbCl₃) or 2 μl of monovalent buffer (negative control) for 40 min at 25° C. For the time course experiments, probing was performed at the indicated times. All reactions were quenched with the addition of 3 μl of 50 mM EDTA pH 8 and precipitated by adding 1/10 volume of Na-Acetate (3M, pH 5.2), 0.51 of glycogen (Invitrogen) and three volumes of ethanol. RNAs were resuspended in 4 μl of loading buffer (82% (v/v) deionized formamide, 0.16% (w/v) xylene cyanol (XC), 0.16% (w/v) bromophenol blue (BB), 10 mM EDTA, pH 8.0) and resolved on a denaturing 5% polyacrylamide gel. The gel was dried, exposed to phosphor screens overnight and scanned using a Typhoon FLA9500 phosphorimager (GE Healthcare) or Typhoon RGB Biomolecular imager (Cytiva).

For Tb³⁺ probing of O.i. and HCV. 1 μg of RNA was diluted in their respective monovalent ion buffers (50 mM HEPES pH7 and 150 mM KCl for O.i. or 50 mM HEPES pH7.2 and 150 mM KCl for HCV) to a final volume of 18 μl. Thereafter, 2N₁ of 100 mM MgCl₂ (final concentration 10 mM) was added and incubated at 37° C. for 30 min. Subsequently, probing was performed by incubating 18 μl of the folded RNA with 2 μl of 10×TbCl₃ stocks prepared in their respective monovalent ion buffers (final 1× concentration from 0.01 mM-2 mM) or 2 μl of respective monovalent buffer (negative control) for 10 min at 25° C. Reactivities were compared under conditions where 0.5 mM TbCl₃ was employed and used in all figures unless indicated otherwise. All reactions were quenched with the addition of 3 μl of 50 mM EDTA pH 8. For the denaturing control, RNA was folded as described elsewhere herein but afterward, deionized formamide was added to a final concentration of 50%. The denatured RNA was probed with a final concentration of 0.5 mM TbCl₃. For the secondary structure control, native gel electrophoresis was carried out as previously described (Su, et al., 2005, Nucleic Acids Res. 33, 6674-6687). RNA was incubated in a monovalent buffer in the absence of MgCl₂ and probing was carried out at the indicated TbCl₃ concentrations. All RNA samples were cleaned up using a Zymo RNA clean and concentrator column according to the manufacturer's instructions.

### Cell Culture of Human RNase P and SARS-CoV-2 Infection

For in-cell studies of RNase P RNA structure, Huh7.5 cells (ATCC) were cultured in Dulbecco's Modified Eagle Medium (DMEM w/o sodium pyruvate) that was supplemented with 10% heat-inactivated fetal bovine serum (FBS) and 1 mM non-essential amino acids. Cells were cultured to ~80% confluency (~5×10⁶ cells) in a 150 cm tissue culture-treated dish.

For studies of SARS-CoV-2 RNA. Huh7.5 cells (ATCC) were cultured in DMEM supplemented with 10% FBS and 1% Penn/Strep. Approximately 5×10⁶ cells were plated in each of the T150 tissue culture-treated flasks and incubated overnight at 37° C./5% CO₂. The next day, media was removed and 5×10⁵ PFU (MOI ~0.1) of SARS-Related Coronavirus 2 Isolate USA/WA2020 (BEI Resources #NR-52281) was added to each flask in fresh media. Cells were incubated with virus inocula until three days post-infection (dpi).

### Cell Lysis Probing

For all flasks the media was aspirated, cells were washed once with cold wash buffer (50 mM HEPES-KOH pH7.2, 150 mM NaCl, 3 mM KCl), and then dislodged in 2 ml of cold wash buffer with a cell scraper. The cells were collected and centrifuged at 200 g×5 min at 4° C. The supernatant was removed and the cells were resuspended in 2 ml lysis buffer (1% TritonX-100, 50 mM HEPES-KOH, pH7.2, 150 mM KCl, 18 mM NaCl, 1 mM MgCl₂, 1 mM CaCl₂), 30 ul SUPERase-In (20 U/μl) and 1× cOmplete Protease Inhibitor Cocktail EDTA-free). To 250 μl of resuspended cells, 50/μl of Turbo DNase (2 U/μl) was added and the mixture was incubated at 37° C. for 20 min. For cell lysis+Proteinase-K probing experiments, cells were prepared, lysed and DNase digested as described elsewhere herein, but the lysis buffer did not contain protease inhibitor. Subsequently, 25 μl of 20 mg/ml Proteinase-K was added to each 250 μl of lysed cells and the mixture was incubated at 37° C. for an additional 20 min.

All reactions were centrifuged at 200 g×15 sec. Probing was performed by incubating 225 μl of supernatant with 25 μl of freshly made 10×TbCl₃ (final 1× concentrations from 0 mM-5 mM, prepared in wash buffer). The reactions were immediately placed on a rocker and allowed to incubate at 25° C. for 10 minutes before quenching with 20 μl of 0.1M EDTA. RNA was extracted using Trizol according to the manufacturer's instructions. For experiments involving RNase P, total RNA was ribosome depleted using a Ribominus kit that was used according to the manufacturer's protocol with the following exception: the ribodepleted supernatant was purified using a Zymo RNA clean and a concentrator to retain RNAs that are greater than 17 nucleotides in size. For experiments involving SARS-CoV-2, total RNA was cleaned using a Zymo RNA clean and concentrator column.

### Reverse Transcription (RT)

For each probing condition, 1-4 μg of in vitro transcribed or cellular RNA was mixed with 1-2 pmol of gene-specific primers (Table 1) and brought to a volume of 7 μl. To anneal primers, the mixture was heated at 90° C. for 1 min followed by 30° C. for 2 min. To initiate reverse transcription, 2 ul of MarathonRT (can be obtained from Kerafast), 10 μl of 2× MarathonRT buffer (100 mM Tris-HCl pH 8.3, 400 mM KCl, 4 mM MgCl₂, 10 mM DTT and 40% glycerol), 1 μl of 10 mM dNTP mix (NEB) were added and incubated at 42° C. for 30 min. RNA was degraded with the addition of 1 μl of 3M KOH, heated to 95° C. for 5 min and snap cooled to 4° C. for 5 min. Thereafter, 1 μl of 3M HCl was added to neutralize the reaction. For primer extension reactions that would be visualized using electrophoresis, reverse transcription was carried out as described, but using a ³²P labeled primer. After the reaction, cDNA was ethanol precipitated at −20° C. overnight. The cDNA pellets were dissolved in 5 μl of loading buffer and resolved on a denaturing 5% polyacrylamide gel. The gel was dried, exposed to phosphor screens overnight and scanned using a Typhoon RGB Biomolecular imager (Cytiva). For ladder generation, RT was carried out using a Thermo Sequenase cycling kit according to the manufacturer's instructions with an input of 500 ng of the template.

Amino acid sequence of Marathon RT (E. r. Group II intron reverse transcriptase:

Accession ID No. CBK92290.1;
Source: Eubacterium rectale):
(SEQ ID NO: 1)
MDTSNLMEQILSSDNLNRAYLQVVRNKGAEGVDGMKYTELKEHLA
KNGETIKGQLRTRKYKPQPARRVEIPKPDGGVRNLGVPTVTDRFI
QQAIAQVLTPIYEEQFHDHSYGFRPNRCAQQAILTALNIMNDGND
WIVDIDLEKFFDTVNHDKLMTLIGRTIKDGDVISIVRKYLVSGIM
IDDEYEDSIVGTPQGGNLSPLLANIMLNELDKEMEKRGLNFVRYA
DDCIIMVGSEMSANRVMRNISRFIEEKLGLKVNMTKSKVDRPSGL
KYLGFGFYFDPRAHQFKAKPHAKSVAKFKKRMKELTCRSWGVSNS
YKVEKLNQLIRGWINYFKIGSMKTLCKELDSRIRYRLRMCIWKQW
KTPQNQEKNLVKLGIDRNTARRVAYTGKRIAYVCNKGAVNVAISN
KRLASFGLISMLDYYIEKCVTC

### Sequencing Library Preparation

The cDNA products from reverse transcription were purified using AMPure XP beads by adding a 1.2× bead to sample ratio and incubating at room temperature for 10 min. The beads were captured using a magnetic rack for 5 min and washed 3 times with 180 μl of fresh 80% ETOH. The beads were air dried for 5 min and resuspended in 12 μl of water to elute the cDNA. Thereafter, 3′ adaptor ligation was performed by mixing 8 μl of purified cDNA with 0.2 μl of 50 μM 3′ adaptor (Table 1), 1 μl of 10 mM ATP, 2 μl of T4 RNA Ligase buffer, 8 μl of 50% PEG 8000. To reduce ligation bias and barcode the RNA, the ligating adapter contained a random hexamer (NNNNNN) at the 5′ end. The mixture was incubated at 25° C. for 16 hours, followed by enzyme deactivation at 65° C. for 15 min. Ligated products were purified with AMPure XP beads using a 1.2× bead to sample ratio. The products were PCR amplified 4-12 cycles with Q5 HF DNA polymerase (NEB) using Illumina TruSeq forward primer and indexed reverse primers (NEB Next multiplex oligos) (Table 1), with cycle times of 98° C. for 10 sec, 62° C. for 45 sec, and 72° C. for 60 sec. PCR products were purified with 1.2× volume of AMPure XP beads. Library concentrations were determined using a Qubit dsDNA HS Assay Kit and a BioAnalyzer High Sensitivity DNA Analysis. Libraries were diluted, pooled and sequenced using a NextSeq 500/550 or NextSeq 2000 platform.

### Tb-Seq Data Analysis

All FASTQ files were processed using Cutadapt (v1.9.1) to remove Illumina adapter sequences and then aligned to the respective RNA sequence using HISAT2 (v2.10). Stop information was extracted using RTEventsCounter.py script. The probability of stop per nucleotide was calculated as the number of stops divided by the sum of the total number of read-through events plus the number of stops (equation (1)). Probabilities were background subtracted against a no-probe control (equation (2)). Only nucleotides that contained more than 10,000 read-throughs were considered. To better compare probing experiments conducted in different contexts, including in vitro and in cell conditions where efficiencies of cleavage might differ, values were normalized to the top 10^{th} percentile of stop rates, then scaled from 1-8 (termed “reactivity,” below, based on Smola, et al., 2015, Nat. Protoc. 10, 1643-1669).

$$P ( stop ) = \frac{{n}_{stop}}{{n}_{stop} + {n}_{read - through}} \qquad ( 1 )$$

$$Reactivity = {P ( stop )}_{treated} - {P ( stop )}_{untreated} \qquad ( 2 )$$

Reactivities were compared under conditions where 0.5 mM TbCl₃ (in-vitro transcribed RNAs) or 1 mM TbCl₃ (cell lysate RNAs) was employed and used in all figures unless indicated otherwise. For the Δ Tb analysis, the reactivity obtained from cell lysate+Proteinase-K probing experiments was subtracted from the reactivity obtained from cell lysate probing experiments (equation (3)). In order to take a conservative approach, a stringent cutoff of 0.5 was implemented to detect strong differences in reactivities.

$$Δ Tb = {Reactivity}_{Cell lysate + Proteinase - K} - {Reactivity}_{Cell lysate} \qquad ( 3 )$$

### Structure and Graphical Display

All secondary structures were visualized and drawn using StructureEditor. All three-dimensional structure renderings were done using PyMOL Molecular Graphics System, Version 1.2r3pre, Schrödinger, LLC. Graphical displays were made using GraphPad Prism 8 or RStudio Version 1.2.5001. Solvent Accessible Surface Area was calculated using POPScomp.

### Example 3: Developing a High Throughput Sequencing-Based Approach to Detect Tb³⁺ Cleavage Sites

To precisely identify tertiary RNA structural elements in a high throughput manner, the present study adapted a Tb³⁺ RNA cleavage assay for accurate single nucleotide detection in an RNA of interest.

In the classical version of this experiment, the RNA of interest is end-labeled with ³²P, probed with Tb³⁺, and the sites of hydrolysis are visualized after electrophoresis of the RNA.

To adapt this assay to a sequencing readout, it was first determined if the expected Tb³⁺ cleavage sites could be detected as termination events upon reverse transcription (RT) with a processive reverse transcriptase, MarathonRT. The D135 ribozyme derived from yeast group II intron aI5γ, which has been extensively characterized using the classical version of Tb³⁺ probing, was used. It was found that reverse transcription stops (FIG. 1C) recapitulate the previously-published Tb³⁺ cleavage pattern (FIG. 1D), thereby validating RT as a tool to detect Tb³⁺-induced cleavage. This approach was then adapted for NGS sequencing. Specifically, a Tb³⁺ cleaved RNA or an untreated RNA is reverse transcribed with a gene specific RT primer containing a 5′ adapter handle. The resulting cDNA is 3′ adapter-ligated and PCR amplified to add Illumina multiplex handles. A sequencing assay using a pipeline for assessing RT termination events was then implemented to quantify termination (FIG. 1E). This sequencing and analysis approach, Tb-seq, recapitulated the previously published D135 Tb³⁺ cut sites (FIGS. 6A-6B).

To better understand whether Tb-seq could be used as a discovery tool for assessing higher order RNA structure in a variety of RNA types, this method was next applied to evaluate the patterns of cleavage in RNAs with well-determined tertiary structures.

### Example 4: Tb-Seq Reveals Well-Folded RNA Tertiary Elements

To benchmark Tb-seq on RNAs that have never been analyzed with Tb³⁺ cleavage before, transcribed RNAs that contain both well-folded RNA tertiary elements and known metal sites were probed in vitro. This study chose a group II intron from Oceanobacillus iheyensis (O.i.) that has been well characterized biochemically and crystallographically. First, reagent concentrations and reaction times were optimized to obtain an ideal reactivity signal and ensure the RNA is not over-cleaved (FIGS. 7A-7B). Tb-seq was performed using a range of Tb³⁺ concentrations from 0.01 mM-2 mM for 10 min in order to evaluate the intensity and location of cleavage patterns ((FIG. 8). The cleavage signal was abolished if the O.i. intron is denatured prior to probing, supporting the interpretation that cleavage signals are indicators of RNA structure. To determine whether secondary structure alone is sufficient to produce the cleavage pattern, the intron was folded only in the presence of monovalent ions, under conditions lacking the magnesium ions known to promote its characteristic tertiary structure (FIG. 9A). It was found that secondary structure was insufficient to establish the signals, supporting the interpretation that Tb-seq signals correspond to sites of tertiary structure. Instead, it was found that at certain Tb³⁺ concentrations (0.5 mM), non-specific cleavage is observed (FIG. 9B). These results demonstrate that a correctly folded intron containing well-defined tertiary elements is required for Tb³⁺ coordination and site-specific RNA cleavage.

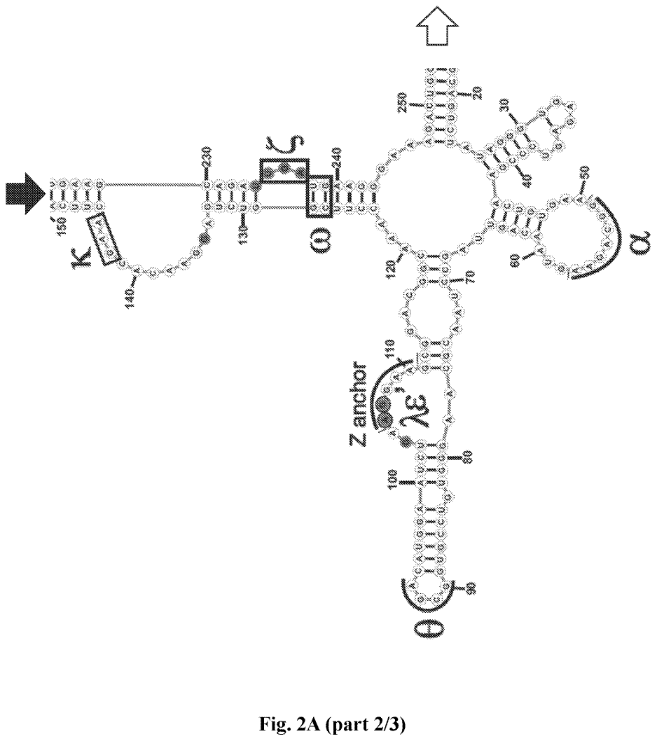

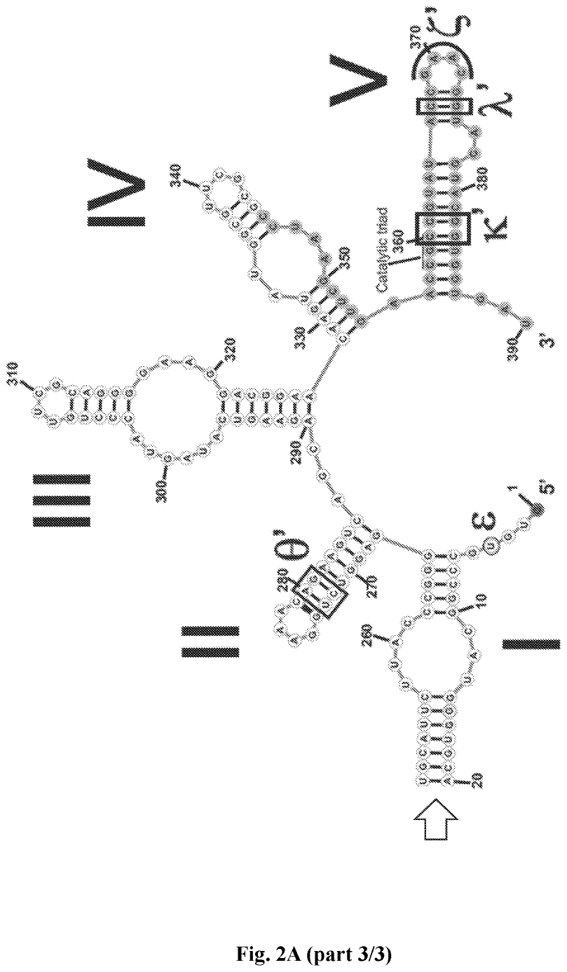

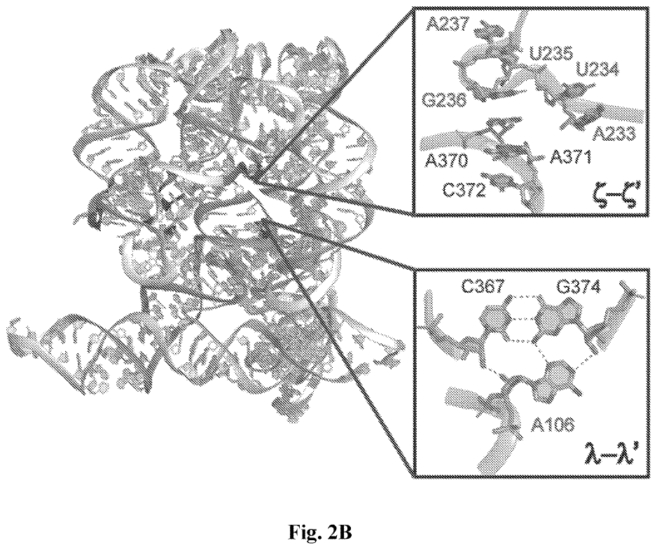

Next, the study established three-point criteria set for selecting nucleotide stop sites that are likely to result from specific, site-bound Tb³⁺-dependent cleavage, which will be called “strong Tb signal”. First, a reactivity value of >0.5 was established for detecting strong sites of cleavage and maximizing probe specificity (see Methods). Second, these sites must be observed in two independent replicates to demonstrate reproducibility. Third, selected sites must show a dependence of signal on Tb³⁺ concentration to ensure that stop signals are not due to spontaneous RT termination events. Nucleotide sites that satisfy these criteria are highlighted in red in the secondary structure diagram of the O.i. intron (FIG. 2B). Upon initial inspection, it was observed that the strongest Tb³⁺ sites are in short loop regions within the RNA secondary structure. Upon close inspection, it became clear that these cleavage sites fall within or are adjacent to the most evolutionarily conserved long-range RNA tertiary interactions that are essential for correctly folding the ribozyme (annotated by Greek letters, FIG. 2B). To further understand the conformation of these sites in 3-D space, the Tb³⁺ signal was visualized on the crystal structure of O.i. intron (FIG. 2A). It was found that Tb³⁺ causes backbone cleavage at regions where the phosphate backbone compresses together to form sharp, stable turns. These turns are all components of RNA tertiary motifs required for the correct folding of the active ribozyme.

To examine the sites of cleavage in greater detail, the study focused on two regions that are specifically recognized by Tb³⁺. The first is the ζ-ζ′ tetraloop-receptor interaction, which is among the best characterized and most important interactions for positioning catalytic intron domain 5 (D5). Here, a single G236 residue in D1 flips out of a sharp backbone turn and base-stacks with A370 in D5 (FIG. 2B, top insert). Several nucleotides (234-237) in the bulge that mediate this ζ-ζ′ tetraloop were identified to contain strong Tb-seq signal. The second motif, λ-λ′ is within the z-anchor, a module that forms multiple higher order structures and serves as a scaffold for properly positioning the 5′ splice site. Notably, strong Tb-seq signal is observed in A106 in D1, which forms a minor groove base triple with nucleotides C267 and G374 in D5 (FIG. 2B, bottom insert). These results demonstrate that Tb³⁺ detects functionally important interactions in group II introns where RNA phosphate backbones come into very close proximity, thereby allowing for multi-helix base stacking and long-range interactions.

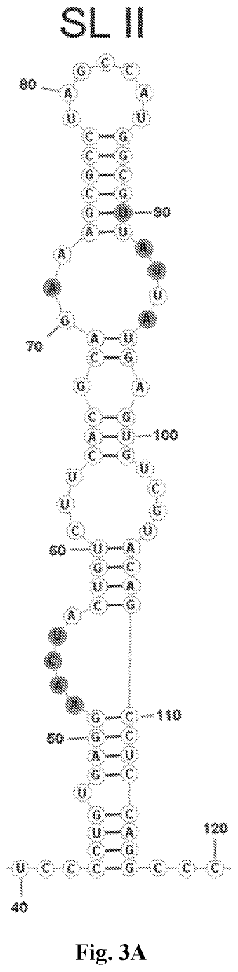

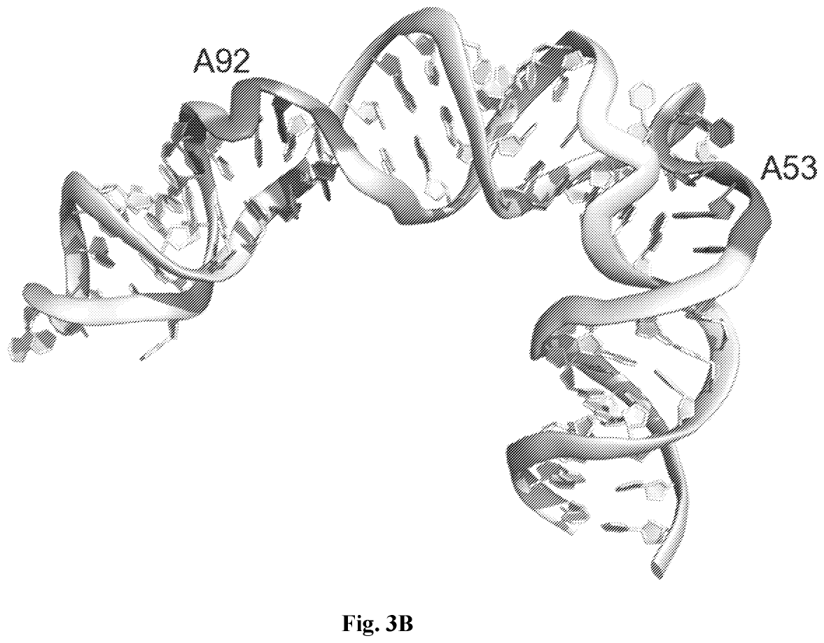

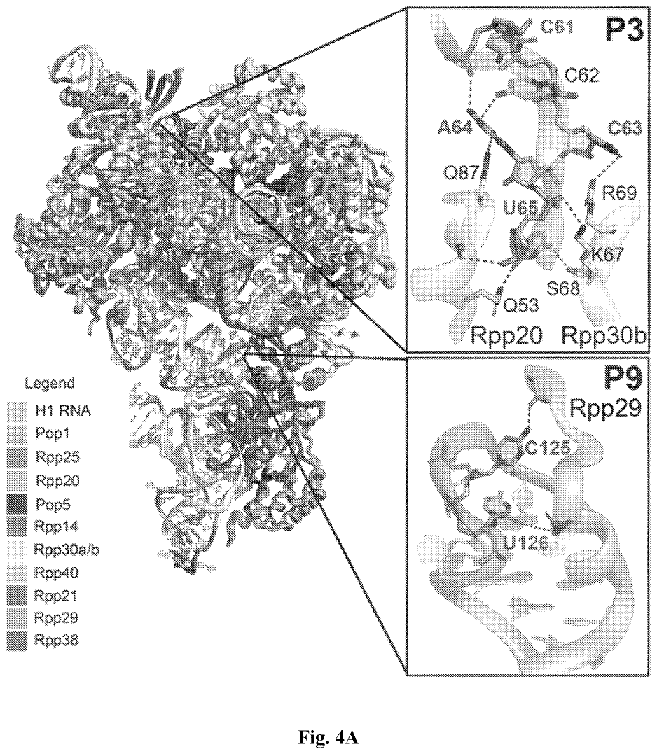

To further test and expand Tb-seq, another class of RNA that contains a well-defined tertiary structure was probed. For this the Hepatitis C Virus (HCV) internal ribosome entry site (IRES), specifically focusing on domain II (which has well-characterized structural features identified by both cryoEM and NMR) was chosen. Implementing the criteria described elsewhere herein, strong Tb³⁺ signal clustering were observed in two regions. The first is a loop region containing nucleotides 92-95, where the phosphate backbones kink and come into close proximity (FIGS. 3A-3B). The second region is near nucleotides 52-54, where the phosphate backbone forms a nearly 90° bend in the RNA (FIGS. 3A-3B). This bend is implicated in positioning of the downstream terminal loop near the 40S E site of the ribosome, which allows for translation of viral proteins. Interestingly, this region has been targeted by functional inhibition studies where multiple small molecules bind and structurally extend the bend into an elongated conformation, inhibiting viral translation. Together, these results indicate terbium probing can detect functionally important structures in RNAs, allowing it to be used as a screening tool for identifying regions that are likely to contain compact motifs.

### Example 5: Tb-Seq Detects Key RNA-Protein Interactions in a Cellular Context

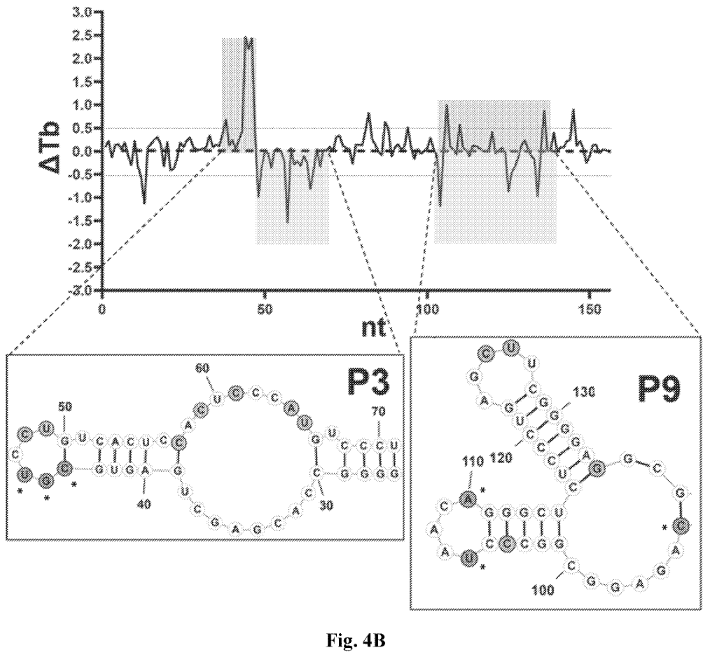

Having established the versatility of Tb-seq on RNAs that have been in-vitro transcribed, the study sought to extend it to cellular contexts, where RNA can fold together with proteins, small molecule ligands and other nucleic acids. The first experiments were conducted on a structurally well-defined cellular RNA with known protein binding sites. To this end, human RNase P was probed in order to understand how terbium can be used to reveal higher order RNA structural motifs in that stable RNP. To circumvent the issues of introducing lanthanide ions into cells, an approach for gently lysing mammalian cells in a way that maintains intact RNA-Protein (RNP) complexes was developed (FIG. 10A). The resulting extract was then treated with Tb³⁺ and the Tb-seq pipeline implemented, using the criteria established for identifying strong sites of specific Tb³⁺ cleavage (FIG. 10B). By comparing the Tb-seq signal with the cryo-EM structure of human RNase P H1 RNA, it was observed that the strongest cleavage sites are found in regions where the RNA backbone bends sharply, notably at the top and bottom of the H1 RNA (nt 47-50 and 169-173; FIG. 4A).

Human RNase P consists of ten protein components that wrap around and bind the H1 RNA at multiple regions, presumably stabilizing its elongated conformation (FIG. 4A). While a number of sites are observed, here are highlight two examples where Tb-seq reveals regions containing critical RNA-protein interactions. The first is a backbone turn located in the loop of stem P9 (FIG. 4A, bottom insert). The bases of nucleotides C125 and U126 form hydrogen bonding interactions with the side chains of the essential core protein, Rpp29. This protein makes multiple contacts with stem P9 and P1, bringing them together in close proximity and stabilizing the downstream helical core of the H1 RNA, which recognizes the 5′ end of pre-tRNA for cleavage. The second site of strong Tb-seq signal is observed in the loop region of stem P3. Here, the backbone, bases, and sugars of the nucleotides targeted by Tb³⁺ (C61, C63, A64, U65), form networks of hydrogen bonds with proteins Rpp20 and Rpp30b (FIG. 4A, top insert). In this context, Tb-seq signals correspond to exposed regions of the RNA which form structural motifs that are stabilized by protein interactions within RNase P.

To further explore the ability of Tb-seq to reveal RNP interactions and to understand the role of the protein in Tb³⁺ detection at these sites, Tb³⁺ was used to probe human RNase P in the absence of proteins. To this end, Tb³⁺ cleavage was conducted on cell lysates that were treated with a proteolytic enzyme (Proteinase-K), which strips proteins from RNA (FIG. 11). As in studies with other chemical probes, a differential reactivity comparison, termed Δ Tb, was then performed to compare changes in H1 RNA structure in the presence and absence of proteins (FIGS. 4B and 11). Consistent with a disruption of a stabilizing protein interaction, the two regions described above become less reactive in the absence of proteins (show a loss in terbium reactivity). By contrast, other nucleotides become more reactive after proteinase K treatment (see stem P3, FIG. 4B), which may result from conformational rearrangement that occurs in the absence of proteins. These data suggest that Δ Tb detects modules of protein-stabilized RNA structures within RNase P, thereby broadening the applicability of this method to probing of RNP interfaces.

### Example 6: Tb-Seq Reveals Modules of Higher-Order Structure in Viral RNAs

Having validated Tb-seq as an RNA tertiary structure probe, the study sought to apply it to discover novel RNA structures in multi-kilobase RNAs, such as long viral RNA genomes. Numerous studies have demonstrated that viral RNA genomes contain secondary and tertiary structures both in the UTRs and coding regions that are important for function. Indeed. Tb-seq was utilized to detect functional RNA structures within the HCV IRES (FIGS. 3A-3B). Given the urgency of detecting functional RNA elements within SARS-CoV-2 RNA and the limited tools available to detect them, cell lysate Tb-seq was performed in SARS-CoV-2 infected cells. The study specifically examined the 5′-terminal 1400 nt of the RNA genome, which contains the 5′UTR, the coding region of Nsp1 and part of the Nsp2 ORF.

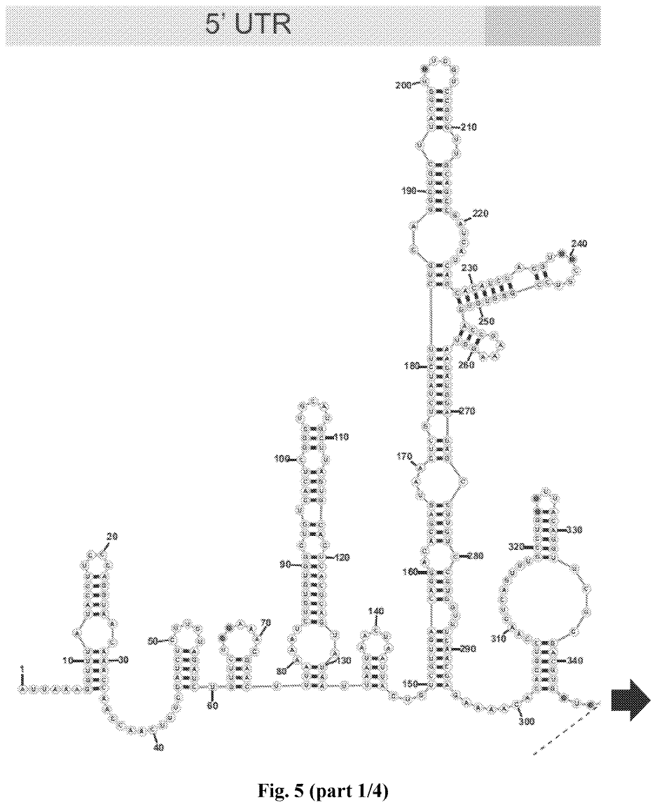

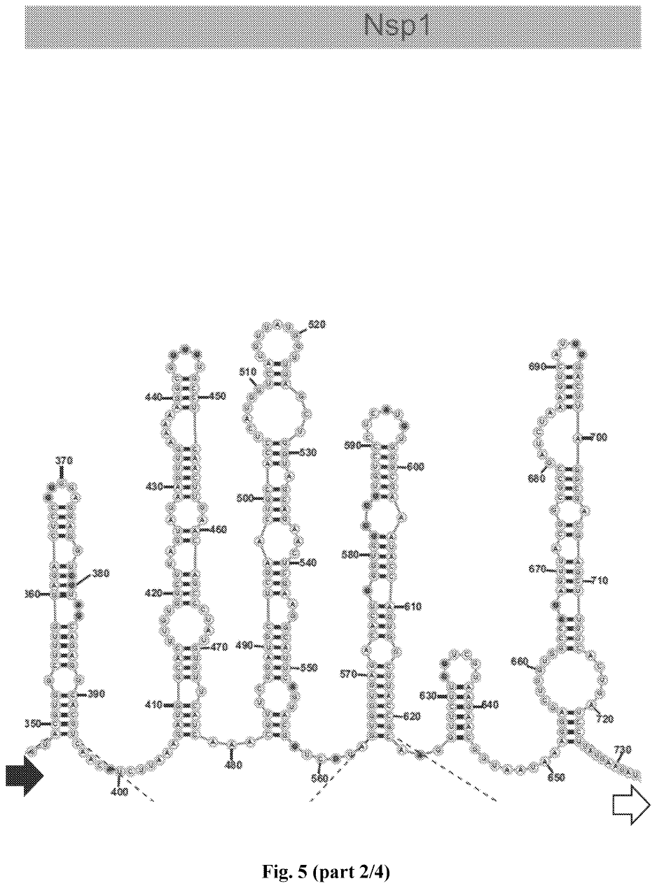

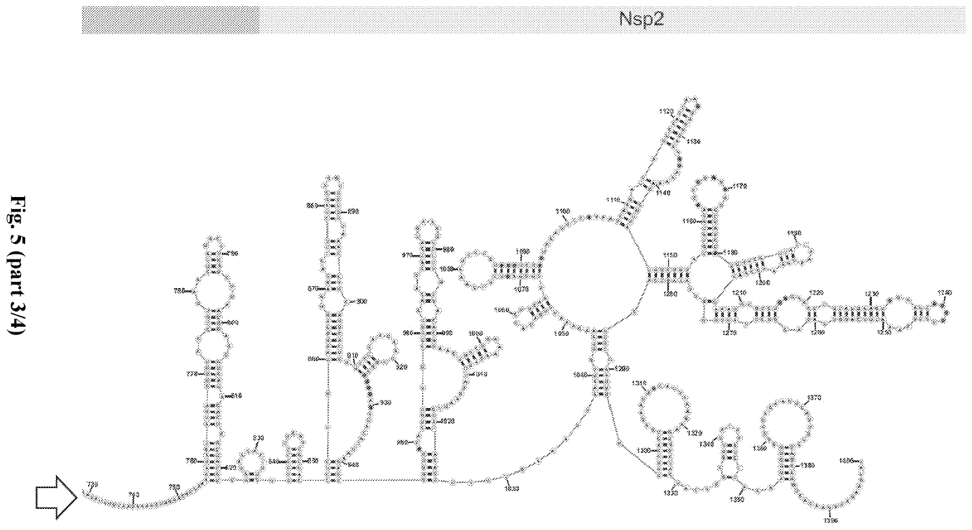

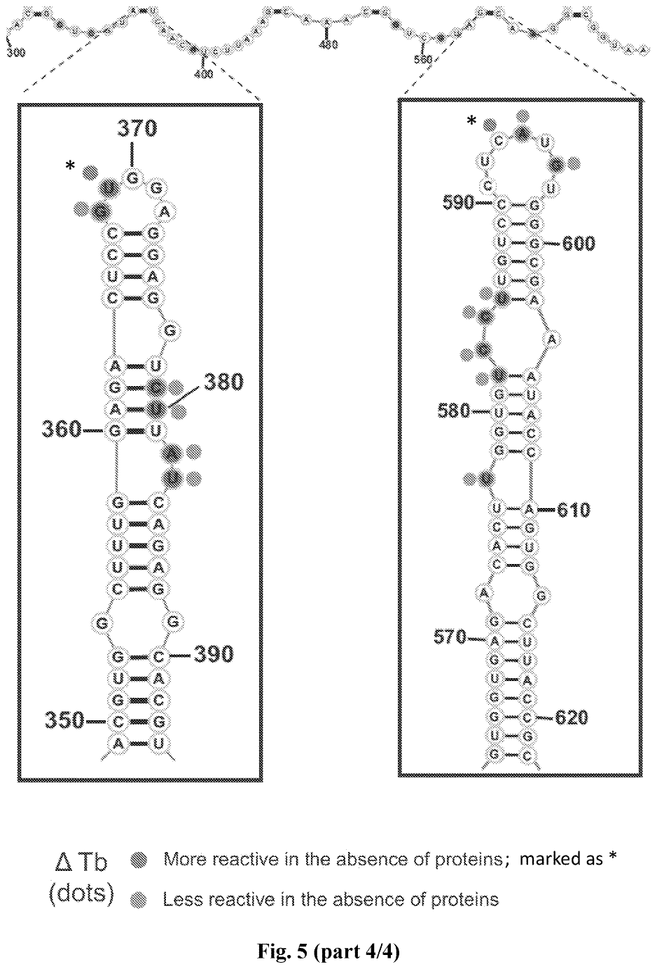

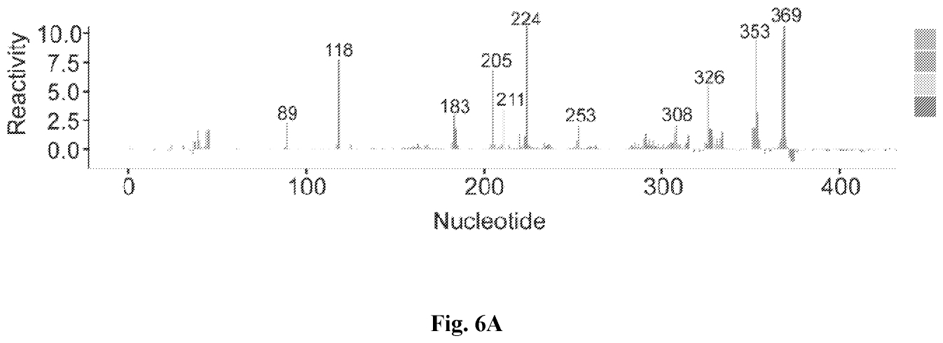

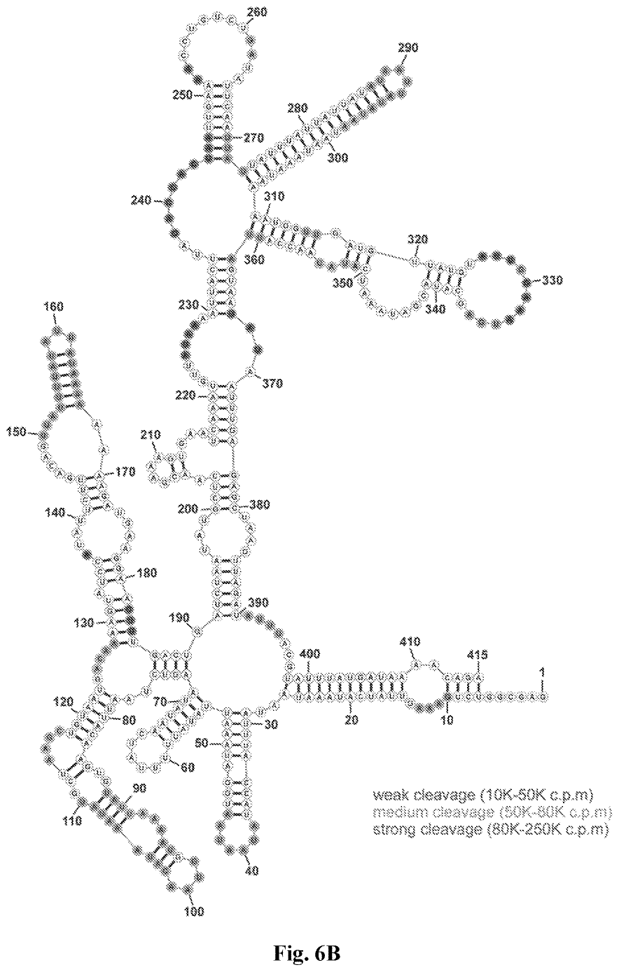

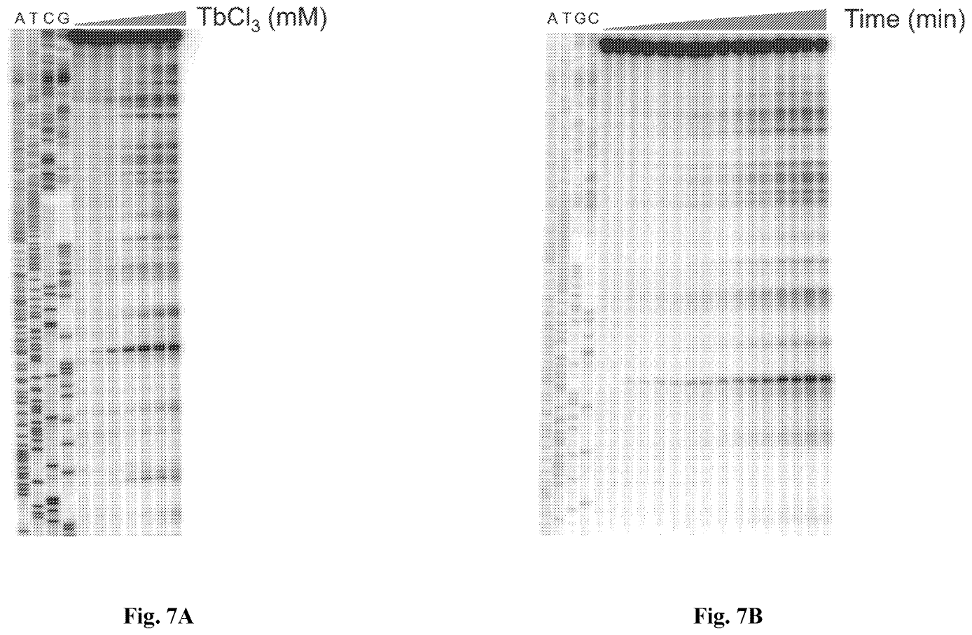

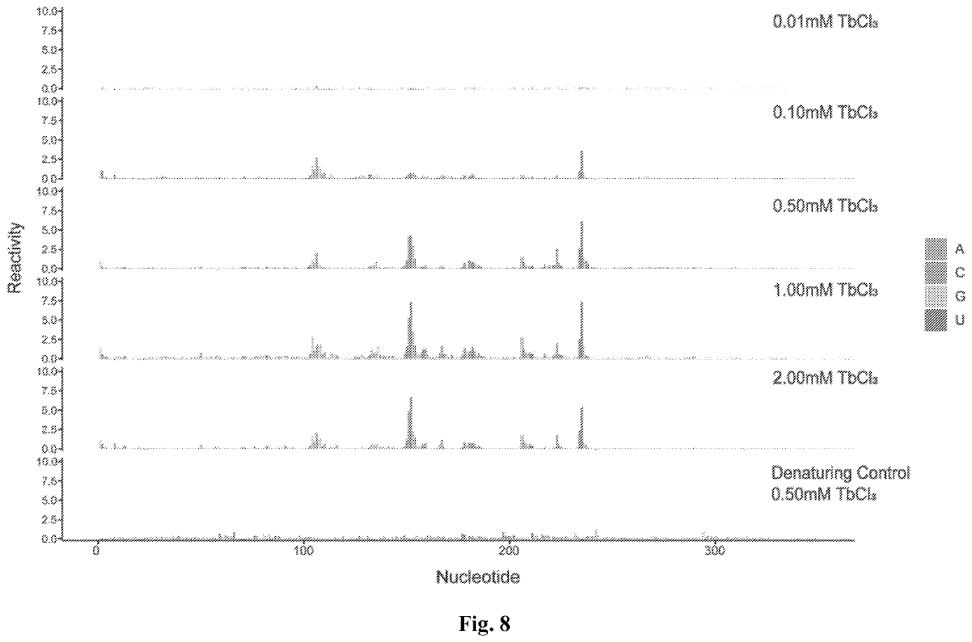

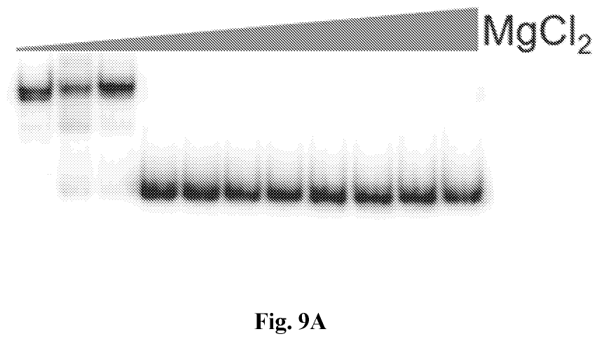

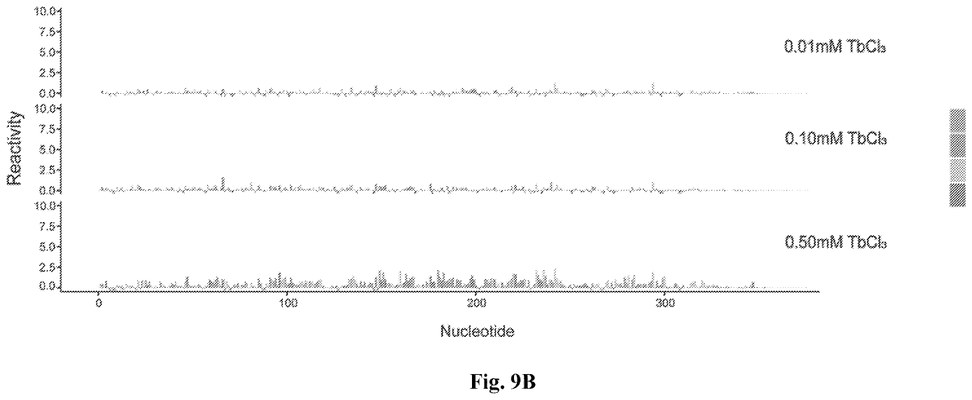

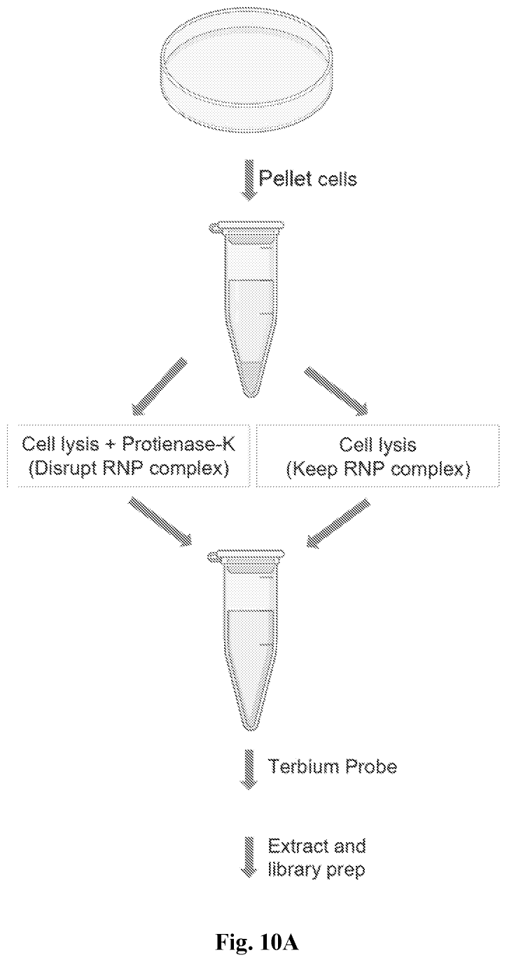

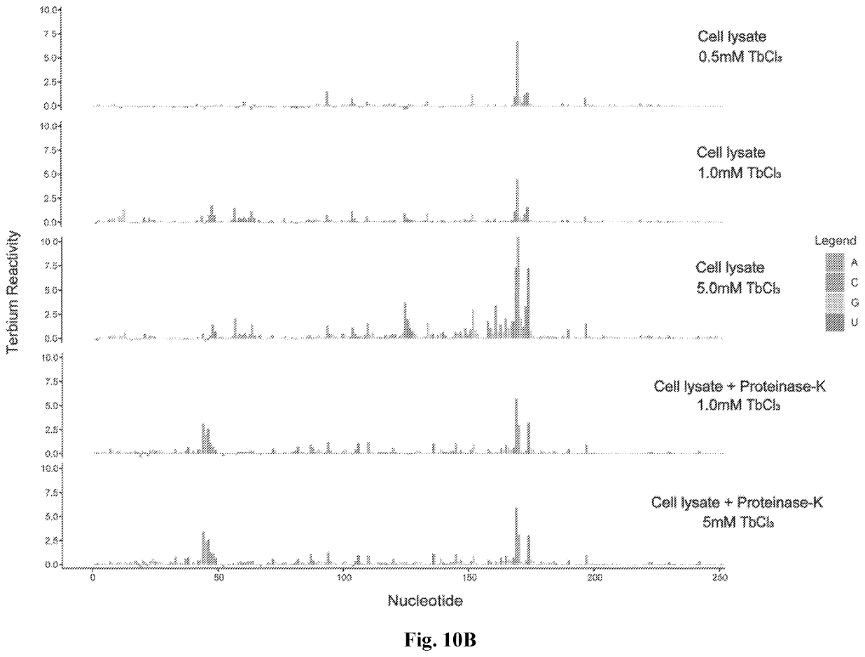

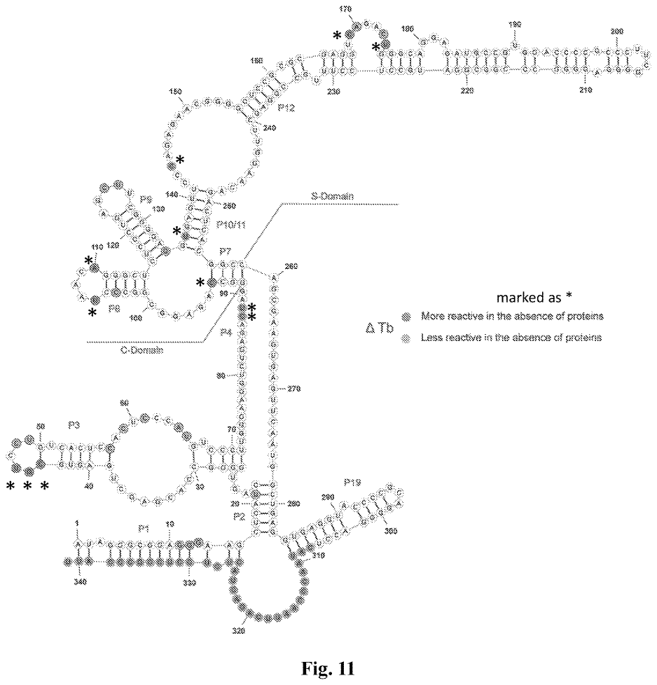

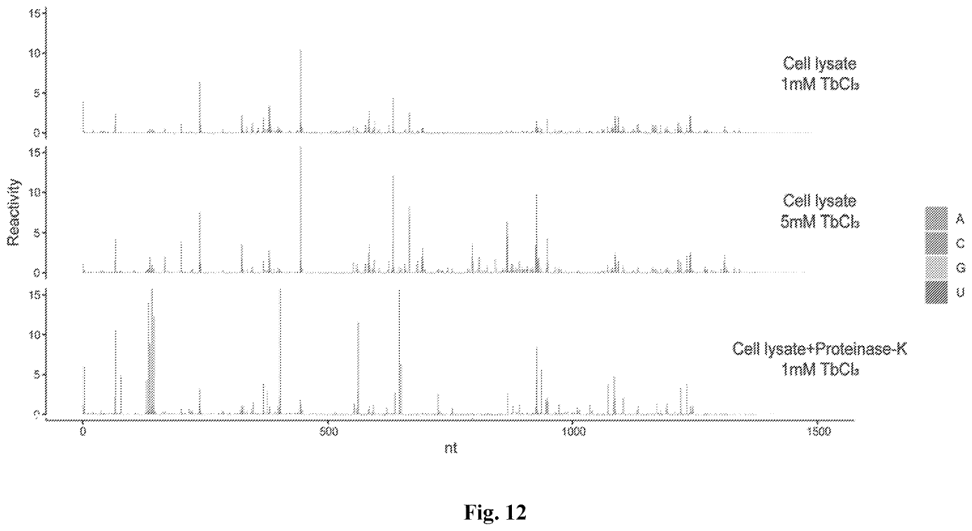

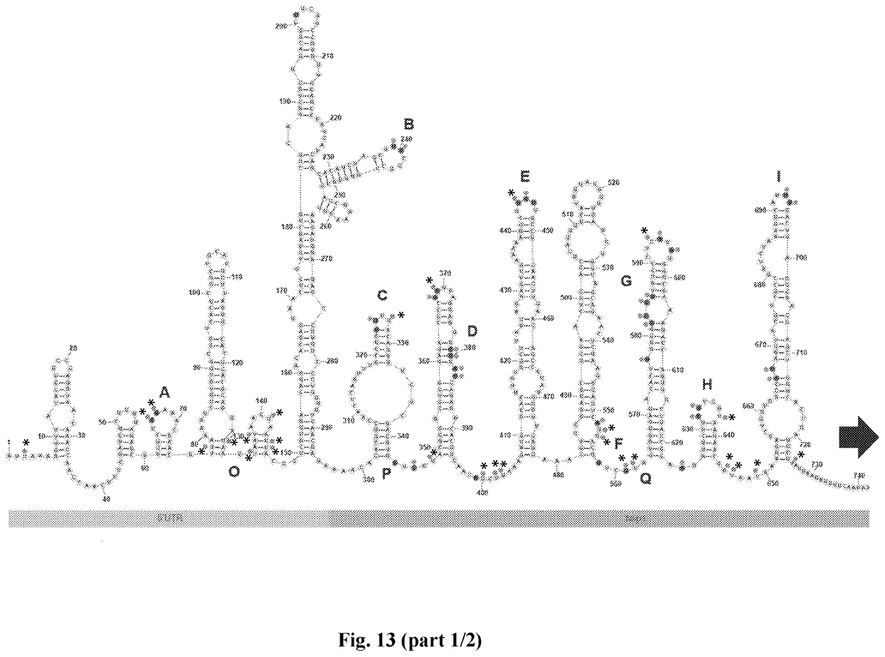

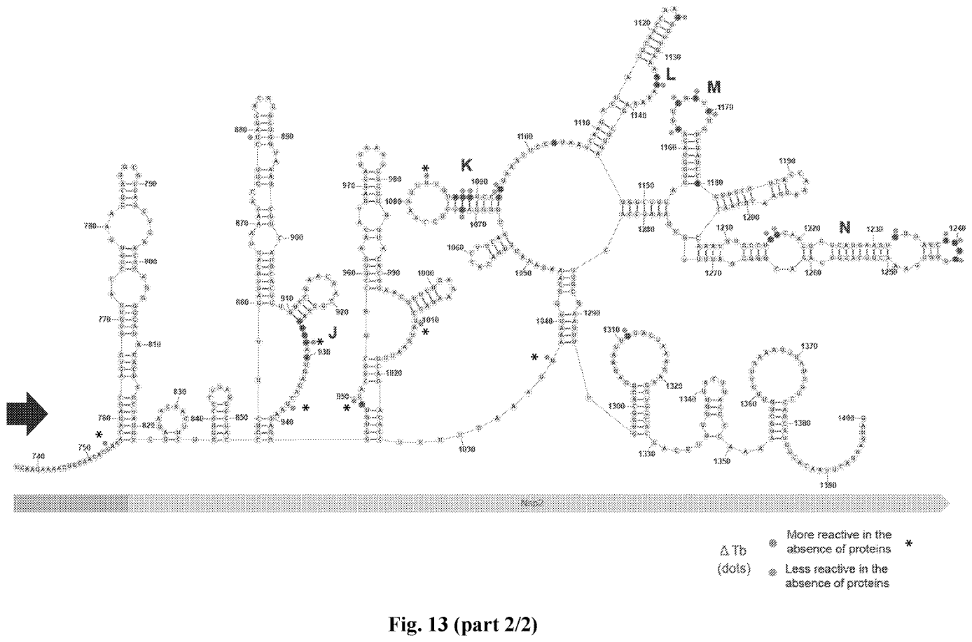

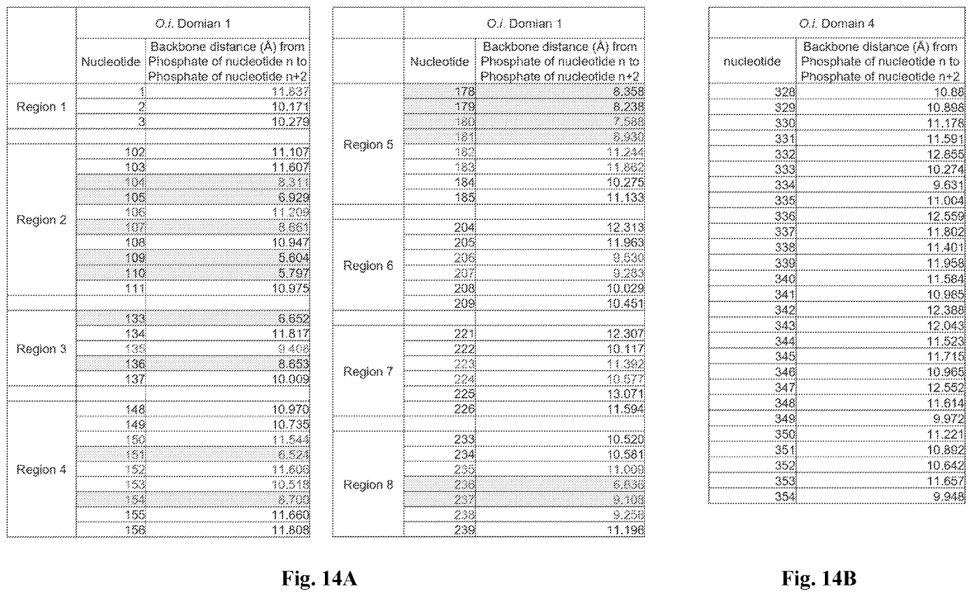

Inspection of the Tb-seq signal profile reveals a distinct cleavage pattern that is characterized by clusters of consecutive cleaved nucleotides (FIG. 12). This signal profile resembles that obtained when probing ribozymes, suggesting a high degree of 3-D structure in the genome. Overlaying these sites onto the predicted secondary structure, strong Tb³⁺ signals were observe in both the UTR and coding region of the genome. Upon closer inspection, the majority of Tb-seq signals were found in small stem loop/bulge regions, implicating these regions as modules of compact RNA structure (FIG. 5).

To further understand the role of protein occupancy on this structured genome and to narrow down sites of potentially functional RNA modules, the study probed in the absence of proteins and implemented the Δ Tb pipeline. Numerous changes are observed in the absence of protein, indicating a global conformational change in the architecture of the genome (FIG. 13). At some sites, the reactivity signal increases, implicating a conformational change in RNA tertiary structure or new backbone accessibility in the absence of proteins. By contrast, there are other sites that become less reactive upon the release of proteins (FIG. 5 inserts). Given the findings with probing RNase P, these sites are likely to represent structural modules containing a sharp backbone bend that is stabilized by protein components. The limited proteomic information on the SARS-CoV-2 genome makes it difficult to assess specific interaction partners. Nevertheless, together these data underscore the utility of combinatorial Tb-seq for narrowing down structural modules and providing a course-grained roadmap of candidate functional elements within a viral genome.

### Example 7

As biologists explore the growing landscape of biologically important multi-kilobase RNAs (such as viral genomes, unprocessed mRNAs, primary miRNAs and long noncoding RNAs), new tools are needed that will enable researchers to focus their attention on specific regions of RNA for detailed functional analysis. The Tb-seq pipeline presented here provides one such filter, yielding valuable information about structurally compact local RNA motifs that differs from the information reflected in other probes of secondary and tertiary structure. In addition, by using the Δ Tb probing strategy, and probing in the presence and absence of protein components, one can narrow down tertiary structures that undergo protein-dependent conformational differences. In certain embodiments, integrating Tb-seq with orthogonal chemical probes, pull-down methods, cross-linking agents, and functional assays can allow for a comprehensive mechanistic understanding of individual RNA molecules.

With recent technological advances, it is now possible to determine high resolution structures of large RNAs. However, multi-kilobase RNAs cannot be visualized in their entirety using these approaches. Most biologically relevant transcripts contain modules of compact structure along with regions that are conformationally flexible. For this reason, most RNAs are amenable to high-resolution structure determination only after careful study of their overall structural landscape. This requires a methodical approach for identifying RNA regions and RNP substructures that can be visualized with powerful tools such as cryo-EM and SAXS. In addition, there are many cases where one must rationally design or isolate stable motifs of RNA and/or RNP complexes. The present study provides a way to identify the most structurally compact regions of a large RNA and, in tandem with other long-range probing methods, choose the best regions for high-resolution investigation.

Performing Tb-seq on RNAs with known structures provided a useful starting point for assessing the types of RNA motifs that are recognized and cleaved by Tb³⁺. The study visualized the structures and noted that most Tb³⁺ cleavage sites occur in regions where multiple phosphate backbone residues pinch together in close proximity. To reflect this, a metric for assessing the “sharpness” of turns in the RNA backbone at Tb³⁺ cleavage sites was computed, deriving the values from high resolution structures of the O.i. intron. Specifically, the backbone phosphate distances between nucleotide n to nucleotide n+2 at sites displaying strong Tb-seq signals (Pₙ→Pₙ₊₂, or every other phosphate) was measured and these data were compared to the corresponding distances in a simple helical structure within domain 4 of the intron (FIGS. 14A-14B). RNA regions with strong Tb-seq signals tended to have very small Pₙ→Pₙ₊₂ values (5.5-9.1 Å) relative to the same distances calculated from a simple helix (9.6-12.5 Å), indicating local compression of the RNA backbone. While some Tb³⁺ cleavage sites, such as those in region 7, are not characterized by small Pₙ→Pₙ₊₂ values, visual inspection of the structure shows that these same nucleotides are part of a larger motif in 3-D space that contains adjacent pinched backbones that are characterized by strong Tb-seq signatures and small Pₙ→Pₙ₊₂ values (in region 5). Therefore, the same bound metal ion may be catalyzing both cleavage events.

The human transcriptome contains a vast set of large, complex RNA molecules, and until recently, the art has lacked the tools to assess their 3-D structural content. However, the biochemical methods that were initially developed to study tRNAs, riboswitches, and ribozymes are being gradually being adapted to explore the growing repertoire of multi-kilobase RNAs that are central to gene expression and pathogenicity. The present study provides a much-needed expansion of the RNA probing toolbox that allows investigators to rapidly pinpoint candidate RNA tertiary structures efficiently and precisely.

TABLE 1
Reverse transcription primers and DNA oligos used in this study.

| Sequence | Description |
|---|---|
| 5′- | Gene specific RT primer for D135 |
| CAGACGTGTGCTCTTCCGATCTTATCACCTATAGTAT | intron. Contains TruSeq overhang. |
| AAGT | |
| -3′ (SEQ ID NO: 2) | |
| 5′- | Gene specific RT primer for O.i. |
| CAGACGTGTGCTCTTCCGATCTATACGGCGGTTCAAG | intron. Contains TruSeq overhang. |
| CTTAGG | |
| -3′ (SEQ ID NO: 3) | |
| 5′- | Gene specific RT primer for |
| CAGACGTGTGCTCTTCCGATCTGGAGGAGAGTAGTCT | human RNase P. Contains TruSeq |
| GAATTGGG | overhang. |
| -3 (SEQ ID NO: 4) | |
| 5′- | Gene specific RT primer for HCV |
| CAGACGTGTGCTCTTCCGATCTTTTTCTTTGAGGTTTA | RNA. Contains TruSeq overhang. |
| GG | |
| -3′ (SEQ ID NO: 5) | |
| 5′- | 1 of 5 gene specific RT primers for |
| CAGACGTGTGCTCTTCCGATCTCCTGTAAAACAGGCA | SARS-CoV-2. Contains TruSeq |
| AACTGAGTTG | overhang. |
| -3′ (SEQ ID NO: 6) | |
| 5′- | 2 of 5 gene specific RT primers for |
| CAGACGTGTGCTCTTCCGATCTCCGTACTGAATGCCT | SARS-CoV-2. Contains TruSeq |
| TCGAG | overhang. |
| -3′ (SEQ ID NO: 7) | |
| 5′- | 3 of 5 gene specific RT primers for |
| CAGACGTGTGCTCTTCCGATCTTAATGCACTCAAGAG | SARS-CoV-2. Contains TruSeq |
| GGTAGC-3′ (SEQ ID NO: 8) | overhang. |
| 5′- | 4 of 5 gene specific RT primers for |
| CAGACGTGTGCTCTTCCGATCTGGTGTCAAATTTCTTT | SARS-CoV-2. Contains TruSeq |
| GCC | overhang. |
| -3′ (SEQ ID NO: 9) | |
| 5′- | 5 of 5 gene specific RT primers for |
| CAGACGTGTGCTCTTCCGATCTCAAGACTATGCTCAG | SARS-CoV-2. Contains TruSeq |
| GTCC | overhang. |
| -3′ (SEQ ID NO: 10) | |
| 5′- | DNA oligo used to ligate 3′-end of |
| Phos-NNNNNNAGATCGGAAGAGCGTCGTGTAG | CDNAs. |
| -3′Bio (N = random nucleotide, SEQ ID NO: 11) | |

### Enumerated Embodiments

The following exemplary embodiments are provided, the numbering of which is not to be construed as designating levels of importance:
Embodiment 1: A method of probing tertiary structure of a ribonucleotide (RNA) molecule, the method comprising:
contacting the RNA molecule with terbium (III) (Tb³⁺) under conditions that allow for Tb³⁺-mediated cleavage of the RNA molecule, thus generating at least one RNA fragment:
determining a sequence of the at least one RNA fragment:
identifying a site of the Tb³⁺-mediated cleavage based on the determined sequence of the at least one RNA fragment; and
identifying a tertiary structure in the RNA molecule based on the identified Tb³⁺-mediated cleavage site.
Embodiment 2: The method of Embodiment 1, wherein the site of the Tb³*-mediated cleavage is within or adjacent to the tertiary structure of the RNA molecule.
Embodiment 3: The method of any one of Embodiments 1-2, wherein the RNA molecule comprises at least 2-100,000 nucleotides.
Embodiment 4: The method of any one of Embodiments 1-3, wherein the sequence of the at least one RNA fragment is determined by capillary electrophoresis or a next generation sequencing (NGS) method.
Embodiment 5: The method of any one of Embodiments 1-4, wherein the at least one RNA fragment is sequenced directly, or wherein the at least one RNA fragment is converted to a DNA molecule and then sequenced.
Embodiment 6: The method of any one of Embodiments 1-5, wherein the at least one RNA fragment is converted to a DNA molecule by a reverse transcriptase (RT) and then sequenced.
Embodiment 7: The method of any one of Embodiments 1-6, wherein the at least one RNA fragment is processed into a cDNA library before being sequenced.
Embodiment 8: The method of Embodiment 7, wherein the at least one RNA fragment or the cDNA molecules in the cDNA library is/are attached with a barcoding sequence.
Embodiment 9: The method of any one of Embodiments 7-8, wherein the determining step comprises aligning the sequence of the cDNA molecules with the sequence of the RNA molecule.
Embodiment 10: The method of any one of Embodiments 1-9, wherein identifying the site of the Tb³⁺-mediated cleavage comprises calculating a stop probability of one or more nucleotide in the RNA molecule, and wherein a nucleotide having a stop probability equal to or higher than a predetermined value indicates that the nucleotide is at the site of the Tb³⁺-mediated cleavage.
Embodiment 11: The method of Embodiment 10, wherein the stop probability per RNA molecule nucleotide is calculated as an abundance of stops for the RNA molecule nucleotide in relative to a sum of the total number of read-through events plus the number of stops for the RNA molecule nucleotide.
Embodiment 12: The method of any one of Embodiments 10-11, wherein the stop probability per RNA molecule nucleotide in the absence of Tb³⁺ contacting is subtracted from the stop probability per RNA molecule nucleotide, thus providing an RNA molecule nucleotide reactivity value.
Embodiment 13: The method of any one of Embodiments 10-12, wherein only the RNA molecule nucleotides having read-throughs equal to or greater than a predetermined number are further considered as potential Tb³⁺-mediated cleavage sites.
Embodiment 14: The method of any one of Embodiments 12-13, wherein the RNA molecule nucleotide reactivity value is normalized to a predetermined value of top percentile of the stop rates for the RNA molecule.
Embodiment 15: The method of Embodiment 14, wherein the normalized RNA molecule nucleotide reactivity value is scaled at a predetermined value.
Embodiment 16: The method of any one of Embodiments 1-14, wherein RNA molecule is contacted with two or more distinct Tb³⁺ concentrations, and wherein the identified tertiary structure in the RNA molecule is based on one or more identified Tb³⁺-mediated cleavage sites that show dependence on Tb³⁺ concentrations.
Embodiment 17: The method of any one of Embodiments 1-16, wherein the method is performed separately with:
(a) the RNA molecule free of a binding partner, and
(b) the RNA molecule in the presence of the binding partner.
Embodiment 18: The method of Embodiment 17, wherein the binding partner is a protein.
Embodiment 19: The method of Embodiment 18, wherein the RNA molecule free of the protein binding partner is generated by removing the protein with a proteolytic enzyme.
Embodiment 20: The method of any one of Embodiments 17-19, wherein the method identifies a tertiary structure induced by the binding partner and/or a tertiary structure disrupted by the binding partner in the RNA molecule, based on a change in a pattern of the Tb³⁺-mediated cleavage between (a) and (b).
Embodiment 21: A kit for probing tertiary structure of an RNA molecule, the kit comprising:
Tb³⁺;
a buffer solution, or a concentrate or substantially pure form thereof, for dissolving the RNA molecule, or diluting a RNA molecule stock solution, and allowing the Tb³⁺ to cleave the RNA molecule in a tertiary structure dependent manner; and
a manual instructing that the RNA molecule is to be cleaved in the buffer solution in the presence of the Tb³⁺
Embodiment 22: The kit of Embodiment 21, wherein
(a) the Tb³⁺ is present in the buffer solution, and the concentration of the Tb³⁺ ranges from about 0.01 mM to about 200 mM in the buffer; or
(b) the Tb³⁺ is provided as a Tb (III) salt or a concentrate thereof, and the manual instructs that the concentration of the Tb³⁺ is to be adjusted to the range from about 0.01 mM to about 200 mM in the buffer.
Embodiment 23: The kit of any one of Embodiments 21-22, wherein the buffer solution, or the concentrate or the substantially pure form thereof, comprises a Good's buffer.
Embodiment 24: The kit of any one of Embodiments 21-23, wherein
(a) a component in the buffer solution ranges from about 0.01 mM to about 200 mM: or
(b) the manual instructs that the concentrate or substantially pure form of the buffer solution is to be prepared so that the component in a buffer solution has a concentration ranging from about 0.01 mM to about 200 mM.
Embodiment 25: The kit of any one of Embodiments 21-24, wherein the kit further comprises a monovalent salt, a divalent salt, or combinations thereof.
Embodiment 26: The kit of Embodiment 25, wherein the concentration of the monovalent salt or the concentration of the divalent salt ranges from about 0.01 mM to about 1M.
Embodiment 27: The kit of any one of Embodiments 21-26, further comprises an RNase inhibitor.
Embodiment 28: The kit of any one of Embodiments 21-27, further comprises a component for preparing an RNA sample for sequencing, wherein the component comprises a barcoding nucleic acid, a ligase, a primer, a reverse transcriptase, a DNA polymerase, an RNase. or combinations thereof.
Embodiment 29: The kit of any one of Embodiments 21-28, further comprises a detergent for lysing the cell.
Embodiment 30: The kit of any one of Embodiments 21-29, further comprising a protease inhibitor, a proteolytic enzyme, or combinations thereof.

1. A method of probing tertiary structure of a ribonucleotide (RNA) molecule, the method comprising:
contacting the RNA molecule with terbium (III) (Tb³⁺) under conditions that allow for Tb³⁺-mediated cleavage of the RNA molecule, thus generating at least one RNA fragment;
determining a sequence of the at least one RNA fragment;
identifying a site of the Tb³⁺-mediated cleavage based on the determined sequence of the at least one RNA fragment; and
identifying a tertiary structure in the RNA molecule based on the identified Tb³⁺-mediated cleavage site.

2. The method of claim 1, wherein at least one of the following applies:
(a) the site of the Tb³⁺-mediated cleavage is within or adjacent to the tertiary structure of the RNA molecule,
(b) the RNA molecule comprises at least 2-100,000 nucleotides,
(c) the sequence of the at least one RNA fragment is determined by capillary electrophoresis or a next generation sequencing (NGS) method,
(d) the at least one RNA fragment is sequenced directly, or wherein the at least one RNA fragment is converted to a DNA molecule and then sequenced,
(e) the at least one RNA fragment is converted to a DNA molecule by a reverse transcriptase (RT) and then sequenced,
(f) the RNA molecule is contacted with two or more distinct Tb³⁺ concentrations, and wherein the identified tertiary structure in the RNA molecule is based on one or more identified Tb³⁺-mediated cleavage sites that show dependence on Tb³⁺ concentrations.

3-6. (canceled)

7. The method of claim 1, wherein the at least one RNA fragment is processed into a cDNA library before being sequenced.

8. The method of claim 7, wherein at least one of the following applies:
(a) the at least one RNA fragment or the cDNA molecules in the cDNA library is attached with a barcoding sequence,
(b) the determining step comprises aligning the sequence of the cDNA molecules with the sequence of the RNA molecule.

9. (canceled)

10. The method of claim 1, wherein identifying the site of the Tb³⁺-mediated cleavage comprises calculating a stop probability of one or more nucleotide in the RNA molecule, and wherein a nucleotide having a stop probability equal to or higher than a predetermined value indicates that the nucleotide is at the site of the Tb³⁺-mediated cleavage.

11. The method of claim 10, wherein the stop probability per RNA molecule nucleotide is calculated as an abundance of stops for the RNA molecule nucleotide in relative to a sum of the total number of read-through events plus the number of stops for the RNA molecule nucleotide.

12. The method of claim 10 wherein at least one of the following applies:
(a) the stop probability per RNA molecule nucleotide in the absence of Tb³⁺ contacting is subtracted from the stop probability per RNA molecule nucleotide, thus providing an RNA molecule nucleotide reactivity value,
(b) only the RNA molecule nucleotides having read-throughs equal to or greater than a predetermined number are further considered as potential Tb³⁺-mediated cleavage sites.

13. (canceled)

14. The method of claim 12, wherein the RNA molecule nucleotide reactivity value is normalized to a predetermined value of top percentile of the stop rates for the RNA molecule.

15. The method of claim 14, wherein the normalized RNA molecule nucleotide reactivity value is scaled at a predetermined value.

16. (canceled)

17. The method of claim 1, wherein the method is performed separately with:
(a) the RNA molecule free of a binding partner, and
(b) the RNA molecule in the presence of the binding partner.

18. The method of claim 17, wherein the binding partner is a protein.

19. The method of claim 18, wherein the RNA molecule free of the protein binding partner is generated by removing the protein with a proteolytic enzyme.

20. The method of claim 17, wherein the method identifies a tertiary structure induced by the binding partner or a tertiary structure disrupted by the binding partner in the RNA molecule, based on a change in a pattern of the Tb³⁺-mediated cleavage between (a) and (b).

21. A kit for probing tertiary structure of an RNA molecule, the kit comprising:
Tb³⁺;
a buffer solution, or a concentrate or substantially pure form thereof, for dissolving the RNA molecule, or diluting a RNA molecule stock solution, and allowing the Tb³⁺ to cleave the RNA molecule in a tertiary structure dependent manner; and
a manual instructing that the RNA molecule is to be cleaved in the buffer solution in the presence of the Tb³⁺.

22. The kit of claim 21, wherein
(a) the Tb³⁺ is present in the buffer solution, and the concentration of the Tb³⁺ ranges from about 0.01 mM to about 200 mM in the buffer; or
(b) the Tb³⁺ is provided as a Tb (III) salt or a concentrate thereof, and the manual instructs that the concentration of the Tb³⁺ is to be adjusted to the range from about 0.01 mM to about 200 mM in the buffer.

23. The kit of claim 21, wherein at least one of the following applies:
(a) the buffer solution, or the concentrate or the substantially pure form thereof, comprises a Good's buffer,
(b) the kit further comprises an RNase inhibitor,
(c) the kit further comprises a component for preparing an RNA sample for sequencing, wherein the component comprises a barcoding nucleic acid, a ligase, a primer, a reverse transcriptase, a DNA polymerase, an RNase, or combinations thereof,
(e) the kit further comprises a detergent for lysing the cell,
(f) the kit further comprises a protease inhibitor, a proteolytic enzyme, or combinations thereof.

24. The kit of claim 21, wherein
(a) a component in the buffer solution ranges from about 0.01 mM to about 200 mM; or
(b) the manual instructs that the concentrate or substantially pure form of the buffer solution is to be prepared so that the component in a buffer solution has a concentration ranging from about 0.01 mM to about 200 mM.

25. The kit of claim 21, wherein the kit further comprises a monovalent salt, a divalent salt, or combinations thereof.

26. The kit of claim 25, wherein the concentration of the monovalent salt or the concentration of the divalent salt ranges from about 0.01 mM to about 1M.

27-30. (canceled)


SEQUENCE LISTING (19 sequences)

SEQ ID NO: 1 - protein, 427 aa, Eubacterium rectale
MDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 2 - DNA, 41 bp, synthetic construct
  source 1..41: mol_type = other DNA
cagacgtgtgctcttccgatcttatcacctatagtataagt

SEQ ID NO: 3 - DNA, 43 bp, synthetic construct
  source 1..43: mol_type = other DNA
cagacgtgtgctcttccgatctatacggcggttcaagcttagg

SEQ ID NO: 4 - DNA, 45 bp, synthetic construct
  source 1..45: mol_type = other DNA
cagacgtgtgctcttccgatctggaggagagtagtctgaattggg

SEQ ID NO: 5 - DNA, 40 bp, synthetic construct
  source 1..40: mol_type = other DNA
cagacgtgtgctcttccgatctttttctttgaggtttagg

SEQ ID NO: 6 - DNA, 47 bp, synthetic construct
  source 1..47: mol_type = other DNA
cagacgtgtgctcttccgatctcctgtaaaacaggcaaactgagttg

SEQ ID NO: 7 - DNA, 42 bp, synthetic construct
  source 1..42: mol_type = other DNA
cagacgtgtgctcttccgatctccgtactgaatgccttcgag

SEQ ID NO: 8 - DNA, 43 bp, synthetic construct
  source 1..43: mol_type = other DNA
cagacgtgtgctcttccgatcttaatgcactcaagagggtagc

SEQ ID NO: 9 - DNA, 41 bp, synthetic construct
  source 1..41: mol_type = other DNA
cagacgtgtgctcttccgatctggtgtcaaatttctttgcc

SEQ ID NO: 10 - DNA, 41 bp, synthetic construct
  source 1..41: mol_type = other DNA
cagacgtgtgctcttccgatctcaagactatgctcaggtcc

SEQ ID NO: 11 - DNA, 28 bp, synthetic construct
  source 1..28: mol_type = other DNA
  modified_base 1: mod_base = OTHER; note = Phosphate group attached to the 5'-end
  variation 1..6: note = Each of the Ns is a random nucleotide
nnnnnnagatcggaagagcgtcgtgtag

SEQ ID NO: 12 - RNA, 81 nt, synthetic construct
  source 1..81: mol_type = other RNA
tcccctgtgaggaactactgtcttcacgcagaaagcgcctagccatggcgttagtatgagtgtcgtacagcctccaggccc

SEQ ID NO: 13 - RNA, 46 nt, synthetic construct
  source 1..46: mol_type = other RNA
ggggccacgagctgagtgcgtcctgtcactccactcccatgtccct

SEQ ID NO: 14 - RNA, 44 nt, synthetic construct
  source 1..44: mol_type = other RNA
cagaggcggccctaacagggctctccctgagcttcggggaggcg

SEQ ID NO: 15 - RNA, 1400 nt, synthetic construct
  source 1..1400: mol_type = other RNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtag

SEQ ID NO: 16 - RNA, 46 nt, synthetic construct
  source 1..46: mol_type = other RNA
acgtggctttggagactccgtggaggaggtcttatcagaggcacgt

SEQ ID NO: 17 - RNA, 59 nt, synthetic construct
  source 1..59: mol_type = other RNA
gtggtgagacacttggtgtccttgtccctcatgtgggcgaaataccagtggcttaccgc

SEQ ID NO: 18 - RNA, 415 nt, synthetic construct
  source 1..415: mol_type = other RNA
gagcggtctgaaagttatcataaataatatttaccatataataatggataaattatatttttatcaatataagtctaattacaagtgtattaaaatggtaacataaatatgctaagctgtaatgacaaaagtatccatattcttgacagttattttatattataaaaaaaagatgaaggaactttgactgatctaatatgctcaacgaaagtgaatcaaatgttataaaattacttacaccactaattgaaaacctgtctgatattcaattattatttattattatataattatataataataaataaaatggttgatgttatgtattggaaatgagcatacgataaatcatataaccattagtaatataatttgagagctaagttagatatttacgtatttatgataaaacaga

SEQ ID NO: 19 - RNA, 341 nt, synthetic construct
  source 1..341: mol_type = other RNA
atagggcggagggaagctcatcagtggggccacgagctgagtgcgtcctgtcactccactcccatgtcccttgggaaggtctgagactagggccagaggcggccctaacagggctctccctgagcttcggggaggtgagttcccagagaacggggctccgcgcgaggtcagactgggcaggagatgccgtggaccccgcccttcggggaggggcccggcggatgcctcctttgccggagcttggaacagactcacggccagcgaagtgagttcaatggctgaggtgaggtaccccgcaggggacctcataacccaattcagactactctcctccgcccatt